(19) United States
(12) Patent Application Publication
(10) Pub. No.: US 2005/0060199 A1
(43) Pub. Date: Mar. 17, 2005

(54) System and method for managing diseases according to standard protocols and linking patients to medication samples and related benefits

(76) Inventor: Louis Siegel, Rochester, NY (US)

Correspondence Address: BASCH & NICKERSON LLP, 1777 PENFIELD ROAD, PENFIELD, NY, 14526, US

(21) Appl. No.: 10/938,981

(22) Filed: Sep. 10, 2004

Related U.S. Application Data
(60) Provisional application No. 60/501,809, filed on Sep. 11, 2003.

(51) Int. Cl. G06F 17/60; A61B 5/00

(52) U.S. Cl. 705/2; 600/300

(57) ABSTRACT

A method and system to enable healthcare providers to utilize computers in chronic disease treatment, and more particularly to the management of chronic diseases in a manner that follows recognized standard-of-care recommendations (SOC) and links the patient to benefit opportunities such as medication samples and other benefits offered by pharmaceutical manufacturers and insurers

### PRIORITY CLAIM

This application claims the benefit and priority of U.S. Provisional Application for Patent Ser. No. 60/501,809, filed Sep. 11, 2003 by Louis Siegel, said application being hereby incorporated by reference in its entirety for its teachings.

### FIELD OF THE INVENTION

The field of the present invention relates to systems and methods to enable healthcare providers to utilize the assistance of computers in chronic disease treatment and management, and more particularly to 1) management of chronic diseases in a manner that follows recognized standard-of-care recommendations (SOC); and 2) link the patient to generic prescribing opportunities and medication samples as well as other benefits offered by pharmaceutical manufacturers and insurers. The present invention may be used as a stand-alone system and method, or with aspects thereof incorporated into an Electronic Medical Record System (EMRS), for example as an icon-selected application (e.g., as in initiation of Microsoft® Word™ in Windows™).

### COMPUTER PROGRAM LISTING APPENDIX

A computer program listing Appendix, including exemplary database files, is submitted on a single compact disc is included and the material thereon is hereby incorporated-by-reference. The total number of compact discs is 1, including 1 original and 1 duplicate, each of which include 180 files as follow:

| NAME | SIZE | CREATED | |
|---|---|---|---|
| accessruntimepath.txt | 57 | Jan. 06, 2004 | 05:52 PM |
| ADODB.dll | 110,592 | Oct. 07, 2003 | 09:47 AM |
| dao.dll | 65,536 | Jan. 01, 2004 | 10:55 PM |
| databasefilepath.txt | 62 | Jan. 02, 2004 | 11:52 PM |
| DBXEZ.exe | 3,174,400 | Aug. 17, 2004 | 06:42 PM |
| DbxEZ.ico | 3,638 | Jun. 08, 2004 | 12:21 AM |
| DbxEz.mdb | 1,249,280 | Aug. 17, 2004 | 06:41 PM |
| DbxEzLookups.mdb | 557,056 | Aug. 17, 2004 | 06:39 PM |
| DbxEzMedications.mdb | 323,584 | Sep. 10, 2004 | 10:03 AM |
| DbxEzReports.mdb | 9,125,888 | Aug. 21, 2004 | 12:56 AM |
| export.ini | 538 | Sep. 10, 2004 | 10:39 AM |
| Form_Form1.cls | 8,302 | Sep. 10, 2004 | 10:08 AM |
| Interop.Access.dll | 966,656 | Mar. 07, 2001 | 09:15 AM |
| Interop.OWC10.dll | 434,176 | Feb. 23, 2001 | 07:36 PM |
| Interop.VBIDE.dll | 57,344 | Jan. 22, 2001 | 08:39 PM |
| list.txt | 0 | Sep. 10, 2004 | 11:31 AM |
| Microsoft.Office.Interop.Access.dll | 974,848 | Sep. 06, 2002 | 01:52 PM |
| Microsoft.Office.Interop.Owc.dll | 438,272 | Jan. 01, 2004 | 10:55 PM |
| Microsoft.Vbe.Interop.dll | 57,344 | Jan. 01, 2004 | 10:55 PM |
| Module1.bas | 3,065 | Sep. 10, 2004 | 10:10 AM |
| MSACC.OLB | 435,616 | Mar. 07, 2001 | 09:15 AM |
| mscomctl.dll | 229,376 | Jan. 01, 2004 | 10:55 PM |
| MSDATASRC.dll | 4,096 | Jan. 01, 2004 | 10:55 PM |
| Office.dll | 196,608 | Jan. 01, 2004 | 10:55 PM |
| OWC10.DLL | 7,436,272 | Feb. 23, 2001 | 07:36 PM |
| QueryACEARBLookup.txt | 18 | Sep. 10, 2004 | 10:40 AM |
| QueryACEARBLookupNULL.txt | 18 | Sep. 10, 2004 | 10:40 AM |
| QueryAdvertisingCoupons.txt | 339 | Sep. 10, 2004 | 10:40 AM |
| QueryAdvertisingMedicines.txt | 307 | Sep. 10, 2004 | 10:40 AM |
| QueryBiguanideLookup.txt | 21 | Sep. 10, 2004 | 10:40 AM |
| QueryBiguanideLookupNULL.txt | 21 | Sep. 10, 2004 | 10:40 AM |
| QueryCalculatedLabDates.txt | 75 | Sep. 10, 2004 | 10:39 AM |
| QueryCO.txt | 234 | Sep. 10, 2004 | 10:39 AM |
| QueryCONULL.txt | 217 | Sep. 10, 2004 | 10:39 AM |
| QueryDateEntry.txt | 36 | Sep. 10, 2004 | 10:39 AM |
| QueryDesktopRecorder.txt | 28 | Sep. 10, 2004 | 10:39 AM |
| QueryGroupAdvertisement.txt | 82 | Sep. 10, 2004 | 10:39 AM |
| QueryLastRecentDate.txt | 34 | Sep. 10, 2004 | 10:39 AM |
| QueryLastRecentDateSubSet.txt | 54 | Sep. 10, 2004 | 10:39 AM |
| QueryLastRecentDateSubSetOLD.txt | 54 | Sep. 10, 2004 | 10:39 AM |
| QueryLoadLabFrequencies.txt | 413 | Sep. 10, 2004 | 10:00 AM |
| QueryLoadLabFrequencies2.txt | 183 | Sep. 10, 2004 | 10:24 AM |
| QueryMedicationClassesAll.txt | 103 | Sep. 10, 2004 | 10:39 AM |
| QueryMedicationClassesAllNULL.txt | 13 | Sep. 10, 2004 | 10:39 AM |
| QueryMedicationClassesINSULIN.txt | 30 | Sep. 10, 2004 | 10:38 AM |
| QueryMedicationClassesINSULINNULL.txt | 12 | Sep. 10, 2004 | 10:38 AM |
| QueryMedicationClassesNONINSULIN.txt | 85 | Sep. 10, 2004 | 10:38 AM |
| QueryMedicationClassesNONINSULINNULL.txt | 13 | Sep. 10, 2004 | 10:38 AM |
| QueryMedicationClassesNONINSULINNULL_OLD.txt | 13 | Sep. 10, 2004 | 10:38 AM |
| QueryMedicationClassesOnACE.txt | 14 | Sep. 10, 2004 | 10:38 AM |
| QueryMedicationClassesOnACENULL.txt | 14 | Sep. 10, 2004 | 10:38 AM |
| QueryMedicationClassesOnARB.txt | 14 | Sep. 10, 2004 | 10:38 AM |
| QueryMedicationClassesOnARBNULL.txt | 14 | Sep. 10, 2004 | 10:38 AM |
| QueryMedicationClassesOnCARDIOVASC.txt | 14 | Sep. 10, 2004 | 10:38 AM |
| QueryMedicationClassesOnCARDIOVASCNULL.txt | 14 | Sep. 10, 2004 | 10:38 AM |
| QueryMedicationClassesOnDM.txt | 14 | Sep. 10, 2004 | 10:38 AM |
| QueryMedicationClassesOnDMNULL.txt | 14 | Sep. 10, 2004 | 10:38 AM |
| QueryMedicationClassesOnINSULIN.txt | 14 | Sep. 10, 2004 | 10:38 AM |
| QueryMedicationClassesOnINSULINNULL.txt | 14 | Sep. 10, 2004 | 10:38 AM |
| QueryMedicationClassesOnLIPID.txt | 14 | Sep. 10, 2004 | 10:38 AM |
| QueryMedicationClassesOnLIPIDNULL.txt | 14 | Sep. 10, 2004 | 10:38 AM |
| QueryMedicationClassesOnOTHER.txt | 14 | Sep. 10, 2004 | 10:37 AM |
| QueryMedicationClassesOnOTHERNULL.txt | 14 | Sep. 10, 2004 | 10:37 AM |
| QueryMedicationCurrent.txt | 871 | Sep. 10, 2004 | 10:37 AM |
| QueryMedicationCurrentACEARB.txt | 231 | Sep. 10, 2004 | 10:37 AM |
| QueryMedicationCurrentACEARBNULL.txt | 169 | Sep. 10, 2004 | 10:37 AM |
| QueryMedicationCurrentCARDIOVASC.txt | 257 | Sep. 10, 2004 | 10:37 AM |
| QueryMedicationCurrentCARDIOVASCNULL.txt | 169 | Sep. 10, 2004 | 10:37 AM |
| QueryMedicationCurrentDM.txt | 361 | Sep. 10, 2004 | 10:37 AM |
| QueryMedicationCurrentDMNULL.txt | 169 | Sep. 10, 2004 | 10:37 AM |
| QueryMedicationCurrentINSULIN.txt | 308 | Sep. 10, 2004 | 10:37 AM |
| QueryMedicationCurrentINSULINNULL.txt | 170 | Sep. 10, 2004 | 10:37 AM |
| QueryMedicationCurrentINSULINold.txt | 307 | Sep. 10, 2004 | 10:37 AM |
| QueryMedicationCurrentLIPID.txt | 233 | Sep. 10, 2004 | 10:37 AM |
| QueryMedicationCurrentLIPIDNULL.txt | 169 | Sep. 10, 2004 | 10:37 AM |
| QueryMedicationCurrentNULL.txt | 179 | Sep. 10, 2004 | 10:37 AM |
| QueryMedicationCurrentOld.txt | 842 | Sep. 10, 2004 | 10:37 AM |
| QueryMedicationCurrentOTHER.txt | 297 | Sep. 10, 2004 | 10:36 AM |
| QueryMedicationCurrentOTHERNULL.txt | 169 | Sep. 10, 2004 | 10:36 AM |
| QueryMedicationLastRecent.txt | 713 | Sep. 10, 2004 | 10:36 AM |
| QueryMedicationLastRecent1.txt | 576 | Sep. 10, 2004 | 10:36 AM |
| QueryMedicationLastRecent2.txt | 190 | Sep. 10, 2004 | 10:36 AM |
| QueryMedicationLastRecent3.txt | 195 | Sep. 10, 2004 | 10:36 AM |
| QueryMedicationLastRecentNULL.txt | 149 | Sep. 10, 2004 | 10:35 AM |
| QueryMedicationLastRecentNULL1.txt | 139 | Sep. 10, 2004 | 10:35 AM |
| QueryMedicationLastRecentNULL2.txt | 139 | Sep. 10, 2004 | 10:35 AM |
| QueryMedicationLastRecentNULL3.txt | 139 | Sep. 10, 2004 | 10:35 AM |
| QueryMedicationLastRecentNULLOLD.txt | 139 | Sep. 10, 2004 | 10:35 AM |
| QueryMedicationLastRecentOLD.txt | 684 | Sep. 10, 2004 | 10:35 AM |
| QueryPatientCensus.txt | 208 | Sep. 10, 2004 | 10:35 AM |
| QueryPatientCensusAll.txt | 657 | Sep. 10, 2004 | 10:56 AM |
| QueryPatientCensusAllOld.txt | 313 | Sep. 10, 2004 | 10:56 AM |
| QueryPatientCensusOLD.txt | 197 | Sep. 10, 2004 | 10:35 AM |
| QueryPatientHistory.txt | 464 | Sep. 10, 2004 | 10:35 AM |
| QueryPatientHistoryNULL.txt | 254 | Sep. 10, 2004 | 10:35 AM |
| QueryPatientID.txt | 105 | Sep. 10, 2004 | 10:57 AM |
| QueryPatientInsulin.txt | 558 | Sep. 10, 2004 | 10:35 AM |
| QueryPatientInsulinNULL.txt | 486 | Sep. 10, 2004 | 10:35 AM |
| QueryPatientLifeStyleOld.txt | 220 | Sep. 10, 2004 | 10:34 AM |
| QueryPatientReadingsOld.txt | 464 | Sep. 10, 2004 | 10:34 AM |
| QueryRecentDate.txt | 34 | Sep. 10, 2004 | 10:34 AM |
| QueryRecentDateNULL.txt | 39 | Sep. 10, 2004 | 10:34 AM |
| QueryRecentDateOLD.txt | 34 | Sep. 10, 2004 | 10:34 AM |
| QueryRiskFactorCount.txt | 318 | Sep. 10, 2004 | 10:34 AM |
| QueryRiskFactorCountNULL.txt | 318 | Sep. 10, 2004 | 10:33 AM |
| QueryRiskFactorSum.txt | 218 | Sep. 10, 2004 | 10:33 AM |
| QueryRiskFactorSumNULL.txt | 218 | Sep. 10, 2004 | 10:33 AM |
| QuerySTILLIS.txt | 134 | Sep. 10, 2004 | 10:33 AM |
| QuerySTILLISNULL.txt | 102 | Sep. 10, 2004 | 10:33 AM |
| QueryTop4LabRecords.txt | 11,262 | Sep. 10, 2004 | 10:32 AM |
| QueryTop4LabRecordsTOPNULL.txt | 0 | Sep. 10, 2004 | 10:31 AM |
| QueryTZDLookup.txt | 15 | Sep. 10, 2004 | 10:31 AM |
| QueryTZDLookupNULL.txt | 15 | Sep. 10, 2004 | 10:31 AM |
| Report_Report1.cls | 148,884 | Sep. 10, 2004 | 10:09 AM |
| Report_Report1_Preliminary.cls | 147,447 | Sep. 10, 2004 | 10:09 AM |
| Report_Report3.cls | 335 | Sep. 10, 2004 | 10:09 AM |
| Report_ReportPatientCensus.cls | 553 | Sep. 10, 2004 | 10:09 AM |
| Report_ReportPatientCensusAll.cls | 548 | Sep. 10, 2004 | 10:09 AM |
| Report_ReportTest.cls | 312 | Sep. 10, 2004 | 10:09 AM |
| Report_SubReportCalendar.cls | 40,211 | Sep. 10, 2004 | 10:10 AM |
| Report_SubReportCheckout.cls | 31,443 | Sep. 10, 2004 | 10:10 AM |
| Report_SubReportControlled.cls | 49,793 | Sep. 10, 2004 | 10:10 AM |
| Report_SubReportSchedule.cls | 28,809 | Sep. 10, 2004 | 10:10 AM |
| schema.ini | 48,389 | Sep. 10, 2004 | 10:40 AM |
| stdole.dll | 16,384 | Jan. 01, 2004 | 10:55 PM |
| tbl_advertising.txt | 735 | Sep. 10, 2004 | 09:38 AM |
| tbl_advertising2.txt | 415 | Sep. 10, 2004 | 10:26 AM |
| tbl_diabetes_meds_brandnames.txt | 168 | Sep. 10, 2004 | 09:39 AM |
| tbl_diabetes_meds_brandnames2.txt | 85 | Sep. 10, 2004 | 10:26 AM |
| tbl_doctors.txt | 177 | Sep. 10, 2004 | 09:40 AM |
| tbl_doctors2.txt | 207 | Sep. 10, 2004 | 10:26 AM |
| tbl_goals.txt | 413 | Sep. 10, 2004 | 09:41 AM |
| tbl_goals2.txt | 402 | Sep. 10, 2004 | 10:26 AM |
| tbl_lookup_diastolic.txt | 2,424 | Sep. 10, 2004 | 09:42 AM |
| tbl_lookup_diastolic2.txt | 776 | Sep. 10, 2004 | 10:26 AM |
| tbl_lookup_feet.txt | 252 | Sep. 10, 2004 | 09:55 AM |
| tbl_lookup_feet2.txt | 52 | Sep. 10, 2004 | 10:26 AM |
| tbl_lookup_inches.txt | 756 | Sep. 10, 2004 | 09:55 AM |
| tbl_lookup_inches2.txt | 115 | Sep. 10, 2004 | 10:27 AM |
| tbl_lookup_labs_frequency.txt | 413 | Sep. 10, 2004 | 09:56 AM |
| tbl_lookup_labs_frequency2.txt | 183 | Sep. 10, 2004 | 10:27 AM |
| tbl_lookup_sponser.txt | 1,130 | Sep. 10, 2004 | 09:57 AM |
| tbl_lookup_sponser2.txt | 552 | Sep. 10, 2004 | 10:27 AM |
| tbl_lookup_sponser_old.txt | 63 | Sep. 10, 2004 | 09:57 AM |
| tbl_lookup_systolic.txt | 4,824 | Sep. 10, 2004 | 09:57 AM |
| tbl_lookup_systolic2.txt | 1,676 | Sep. 10, 2004 | 10:27 AM |
| tbl_lookup_visits_frequency.txt | 563 | Sep. 10, 2004 | 09:58 AM |
| tbl_lookup_visits_frequency2.txt | 258 | Sep. 10, 2004 | 10:27 AM |
| tbl_lookup_waist.txt | 1,953 | Sep. 10, 2004 | 09:58 AM |
| tbl_lookup_waist2.txt | 295 | Sep. 10, 2004 | 10:27 AM |
| tbl_lookup_weight.txt | 18,963 | Sep. 10, 2004 | 09:58 AM |
| tbl_lookup_weight2.txt | 3,229 | Sep. 10, 2004 | 10:27 AM |
| tbl_medications.txt | 332,576 | Sep. 10, 2004 | 10:01 AM |
| tbl_medications2.txt | 32,461 | Sep. 10, 2004 | 10:27 AM |
| tbl_medication_version.txt | 113 | Sep. 10, 2004 | 10:01 AM |
| tbl_medication_version2.txt | 56 | Sep. 10, 2004 | 10:27 AM |
| tbl_patienthistory.txt | 249 | Sep. 10, 2004 | 10:16 AM |
| tbl_patienthistory_old.txt | 6,126 | Sep. 10, 2004 | 10:17 AM |
| tbl_patientid | 103 | Sep. 10, 2004 | 10:59 AM |
| tbl_patientid.txt | 320 | Sep. 10, 2004 | 10:58 AM |
| tbl_patientlabs.txt | 3,713 | Sep. 10, 2004 | 10:19 AM |
| tbl_patientlabs_old.txt | 333,127 | Sep. 10, 2004 | 10:20 AM |
| tbl_patientlifestyle.txt | 87 | Sep. 10, 2004 | 10:20 AM |
| tbl_patientlifestyle_old.txt | 3,059 | Sep. 10, 2004 | 10:20 AM |
| tbl_patientmedicines.txt | 773 | Sep. 10, 2004 | 10:20 AM |
| tbl_patientmedicines_old.txt | 28,948 | Sep. 10, 2004 | 10:20 AM |
| tbl_patientreadings.txt | 1,713 | Sep. 10, 2004 | 10:21 AM |
| tbl_patientreadings_old.txt | 155,127 | Sep. 10, 2004 | 10:21 AM |
| tbl_patient_insulin.txt | 511 | Sep. 10, 2004 | 10:21 AM |
| tbl_patient_insulin_old.txt | 5,042 | Sep. 10, 2004 | 10:21 AM |
| tbl_patient_visits.txt | 3,013 | Sep. 10, 2004 | 10:22 AM |
| tbl_patient_visits_old.txt | 258,655 | Sep. 10, 2004 | 10:22 AM |
| tbl_print_session.txt | 19 | Sep. 10, 2004 | 10:22 AM |
| tbl_report_version.txt | 56 | Sep. 10, 2004 | 10:22 AM |
| tbl_users.txt | 2,592 | Sep. 10, 2004 | 09:59 AM |
| tbl_users2.txt | 684 | Sep. 10, 2004 | 10:23 AM |
| tbl_version_application.txt | 66 | Sep. 10, 2004 | 10:23 AM |
| tbl_version_lookup.txt | 113 | Sep. 10, 2004 | 09:59 AM |
| tbl_version_lookup2.txt | 62 | Sep. 10, 2004 | 10:23 AM |
| VBE6EXT.OLB | 45,056 | Jan. 22, 2001 | 08:39 PM |

### COPYRIGHT NOTICE

A portion of the disclosure of this patent document contains material that is subject to copyright protection. The copyright owner has no objection to the facsimile reproduction by anyone of the patent document or the patent disclosure, as it appears in the Patent and Trademark Office patent file or records, but otherwise reserves all copyright rights whatsoever.

### BACKGROUND AND SUMMARY

The rational for the present invention stems from a practice scenario where various patients with diabetes are seen regularly. Chronic diseases, particularly diabetes are increasing in incidence and prevalence. At the same time, the options to treat diabetes are increasing in number and complexity, fueling diversity of care. There are more drugs from which the physician must choose and more parameters to follow. Taken all together, the proper treatment and long term management of diabetes is increasingly complex and office-time critical causing it to become very difficult for the primary care provider to do well without the benefit of a ‘care tool’. Furthermore, diabetes care is non-uniform across all providers because there is no common or universal method to follow or tool for use by all providers. The present invention seeks to address this opportunity and to provide a tool by which a common treatment methodology can be implemented for diabetes or any chronic disease treatment.

The number of specialists, such as endocrinologists, available to treat diabetic patients is declining, thereby putting greater pressure on the primary care provider to treat diabetes, and to treat diabetes well. One result is an increasing management complexity for the treating physician, resulting in diminished care and increasing complications and expense for the patient. The present invention is intended to provide a knowledge base of treatment protocol in the American Diabetes Association (ADA), and/or other standard of care (SOC) recommendations, to all providers in a novel and easy to use system/method that is presently called DbxEZ™. The system automates the healthcare professional user's interaction with patients and thereby minimizes the complexity of diabetes care and, its use encourages conformity to standards of care and similar protocols. Accordingly, the present invention is distinguishable from an electronic medical records system (EMR), as such systems create and fuel care diversity by providing data templates that individual health care providers can create or modify individually. Therefore, not only is it unlikely any two templates would look alike, there is little assurance that such systems would set forth treatment methodologies matching any standard of care.

The optimal care of the chronic disease (e.g., diabetic) patient requires not only complete knowledge of the disease process, all of the available treatment modalities and standards of care, but, requires the ability and time to implement that knowledge and organize and deliver treatment in what is often a brief, office visit setting. The present invention supplements the provider's knowledge by providing a system to conduct care that conforms to standard of care recommendations. The care of the diabetic patient, for the provider, is as much a time and data management challenge as it is a knowledge challenge. Inadequate care results in a growing number of patients experiencing the complications of diabetes with concomitant increases in morbidity and cost. No computer programs are known to provide standardized care in a package suitable for use in a brief office visit. The present invention, by “steering” and conducting the patient office visit, while conforming to SOC, provides standardized care in a package suitable for use in a brief office visit by any healthcare provider.

The increasing number of and cost of diabetes medicines and supplies suggests a closer relationship between the patient, insurers and the suppliers of those medications and supplies may be beneficial to at least the patient, if not also the insurers and manufacturers. By connecting insurers and drug manufacturers to a patient, at the time of the visit, via the PatientMedLink™ (or PatientLink™) described below, the present invention provides a conduit whereby generic prescribing and incentives such as rebates, samples, trial offers and price comparisons, as well as educational and support information can be automatically and accurately delivered to each patient at each office visit.

In 1999, approximately 450,000 deaths occurred among people with diabetes aged 25 years and older representing 19% of all deaths in the U.S. in that age group. Diabetes was the sixth leading cause of death listed on U.S. death certificates. Diabetes is the leading cause of new cases of blindness among adults 20 to 74 years old. Diabetes is the leading cause of treated end-stage renal disease, accounting for 43 percent of new cases. In 1999, a total of 114,478 people with diabetes underwent dialysis or kidney transplantation. More than 60 percent of nontraumatic lower-limb amputations in the U.S. occur among people with diabetes. The American Diabetes Association (ADA) estimates the total (direct and indirect) cost of diabetes in the U.S. in 2000 was $132 billion.

According to the National Diabetes Information Clearing House, for the year 2000, 17 million people, or 6.2 percent of the population have diabetes. Of those, 11.1 million are diagnosed, while 5.9 million are undiagnosed. It has been further estimated that 70% of those diagnosed do not receive care meeting the standards of the American Diabetes Association. By providing a standard platform (e.g., DbxEZ™) upon which all providers can deliver the same contextual care the present invention is believed capable of reducing that percentage.

Although there are many published recommendations for standards of care for diabetes, for example the American Diabetes Association (ADA) Standards of Care (SOC) for Patients With Diabetes Mellitus or the American Association of Clinical Endocrinologists, there are no presently known computerized platforms for the implementation of those recommendations. Moreover, no programs are known to link, at the time of an office visit, the patient to generic prescribing opportunities, samples, prescription vouchers, rebates, discounts, educational or other diabetes support and information.

The present invention, by contrast to an EMR, is a ‘disease specific’, Electronic Medical Encounter™ (EME™), system of application programs. As will be described in more detail below, the present invention “maps” the patient onto accepted care or treatment protocols without any knowledge of the standards of care (SOC) or template construction required by the provider. Moreover, the invention may be embedded within an EMR system, enabling the common use of patient data. Accordingly, the present invention provides a novel format for collection of patient parameters, presentation and access to SOC, and acts as an aid to the healthcare professional to efficiently work with patients having diabetes.

For example the American Diabetes Association and the American Society of Endocrinologists have recommendations for the care and management of diabetes. However, these organizations leave it up to the healthcare provider to read and implement those recommendations in an office visit setting. Physicians are, in most cases, too busy and generally too computer unsophisticated to create the code or content needed to implement those recommendations within the EMR. Therefore, diabetes care rarely regularly conforms to the recommended standards of care.

Further to the objects and advantages described above, the present invention comprises a collection of methods, schemes, logic, displays, computer code and formats effected, in one embodiment, through computer programs to enable and facilitate health care providers to conduct office visits with the affected patients and manage patients with specific diseases by using the framework of one or more accepted standards of care for that disease. In one embodiment, the disease is diabetes and the standards are the standards of the American Diabetes Association. The present invention also links health care providers, patients and manufacturers, drug benefit providers or pharmaceuticals manufacturers together, to deliver educational and product cost-benefits. These may be for drugs the patient takes, and are offered directly to the patient. Moreover, the opportunity to share such benefits and information occurs at the time of each office visit in accordance with an aspect of the present invention.

The absence of a link to pharmaceutical samples and other manufacturer benefits or information, as the present invention enables, results in a patient being deprived of an organized and consistent way to learn of and access these opportunities. Without the present invention a healthcare provider is unlikely to take the time to offer patients such benefits; this is because the provider must be aware of what benefits are available and literally get up, collect and dispense them to the patient. As a more efficient alternative, the present invention automatically identifies which benefits are appropriate and indicates such on the patient's checkout form—so they may be given to the patient automatically.

Currently, drug manufacturers prepare incentive programs to encourage physicians to start patients on and be maintained on their products. There are valid health benefits and economic reasons why physicians and patients should participate in such opportunities. Theses incentive programs can significantly reduce the cost of medications to patients, if used regularly. The present invention further provides a unique way to enable the incentive programs to be used more regularly.

Currently the main connection between the manufacturer and the patient is the physician, and the main connection between the manufacturer and the physician is the pharmaceutical representative. As a rule, physicians are unwilling to spend any more then a token amount of time with the representative, and, as a result, patient-beneficial incentives frequently go unrecognized and unused. The present invention greatly minimizes or eliminates the time needed by the physician to access and utilize the incentive/sample programs—to the benefit of the patient. To accomplish this feature, the present invention uses software that ‘links’ physician pre-approved product use incentives to the patient at the time of the office visit, without physician involvement each time (e.g., given at check-out, automatically).

In accordance with the present invention, there is provided a system, format and method to enable healthcare professionals to utilize a computer system to provide treatment to patients with a chronic disease according to a standard of care protocol, comprising: enabling, in accordance with pre-programmed code, collection and entry of patient parameters associated with the standard of care protocol into a computer system during a visit; storing the patient parameters in a database; using entered patient parameters, automatically generating a patient report card, said report card indicating at least information pertaining to the patient parameters; automatically printing, for the patient, a disease-management calendar showing a future visit and any lab date; and automatically printing, for the patient, a complete medication schedule, wherein the schedule includes any changes to medication in accordance with the healthcare professional's recommendation.

In accordance with another aspect of the present invention, there is provided a method to enable healthcare professionals to provide patients with access to patient benefits during treatment of a chronic disease according to a standard of care protocol, comprising: enabling, in accordance with pre-programmed code, collection and entry of patient parameters associated with the standard of care protocol into a computer system during a visit; storing the patient parameters in a database; using entered patient parameters, automatically identifying pharmaceutical benefits available to the patient and appropriate for the patient's treatment goals; and using entered patient parameters, automatically generating a patient report card, said report card indicating at least information pertaining to the patient parameters. It will be further appreciated that the database of the present invention may be a self-contained database, used in accordance with the method set forth above. Alternatively, at least a portion of the database may be implemented in accordance with an EMR system, so as to provide a single source of patient data (patient history, prior medical treatment, lab results, etc.).

### BRIEF DESCRIPTION OF THE DRAWINGS

FIG. 1 is a schematic illustration of an embodiment of the present invention;

FIG. 2 is a data flow diagram illustrating the general flow of information in accordance with an aspect of the present invention;

FIGS. 3-8B are illustrative examples of user-interface screens depicted on the user computer of FIG. 1 in accordance with an embodiment of the present invention;

FIGS. 9A-9D are exemplary representations of a patient report card document as provided to a patient in accordance with an aspect of the present invention;

FIGS. 10A-10B are examples of a provider chart document generated in accordance with the present invention, and FIG. 10C is an alternative example of the document of FIG. 10B; and

FIGS. 11A-11G are illustrative examples of administrative interface screens available in accordance with an embodiment of the present invention.

The present invention will be described in connection with a preferred embodiment, however, it will be understood that there is no intent to limit the invention to the embodiment described. On the contrary, the intent is to cover all alternatives, modifications, and equivalents as may be included within the spirit and scope of the invention as defined by the appended claims.

### DETAILED DESCRIPTION

For a general understanding of the present invention, reference is made to the drawings. In the drawings, like reference numerals have been used throughout to designate identical elements.

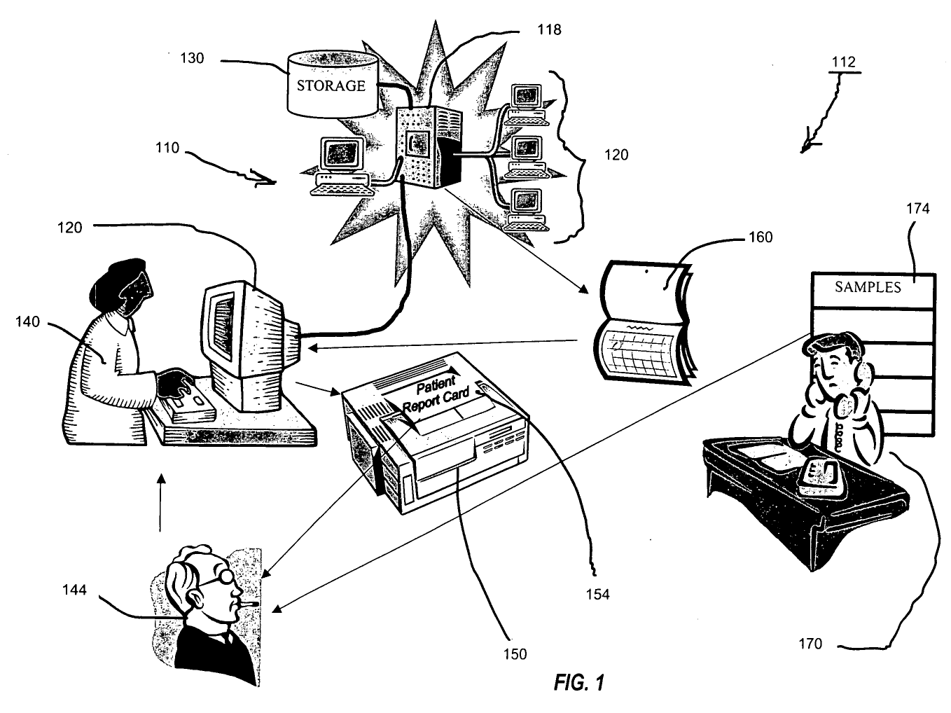

As depicted in FIG. 1, an exemplary embodiment of the present invention is a user computer in a medical professional office. Although it is entirely possible to implement the present invention on a stand-alone workstation, the embodiment of FIG. 1 illustrates a networked computer system as will now be described in more detail. It will be appreciated that some aspects of a networked implementation (e.g., centralized data storage and backup, linked to EMR system, etc.) may prove advantageous. Alternatively, it will be appreciated that a laptop, personal digital assistant (i.e., electronic handheld information device) or similar personal computer device may be employed to implement some or all aspects of the present invention.

Referring to FIG. 1, there is depicted a computer network 110 in a medical practice 112. Network 110 includes a server 118 and a plurality of workstation clients 120. Network 110 also includes a storage device(s) 130 such as a hard disk or equivalent for the storage of at least the patient information. The information, as will be discussed in more detail below, is stored in one or more databases and associated tables and records therein, including partial or complete integration with an electronic medical records system. It will be appreciated that conventional hardware and software may be employed to implement the network, and that the network communications may be accomplished via hard-wired or wireless connections between the components, or a combination thereof.

As will be described in more detail below, the present invention operates to assist, but not replace, a healthcare professional 140 in the treatment and or mitigation of a disease for a patient 144. The professional would interview or discuss the disease, symptoms, test results, measurements, etc. (patient parameters) with the patient and would enter the parameter values directly into a computer system such as network workstation 120. The professional's recommendations (including but not limited to prescriptions, treatments, recommended consultations, etc.) could then be recorded and printed out at printer 150 for the patient, including treatment goals and status on a patient report card 154. As will be shown in detail below, the system may automatically interface with the office calendar system 160, represented by 160, and the information pertaining to a subsequent visit may be depicted on the report card 154.

Furthermore, the present invention is particularly directed to facilitate the sharing or information and benefits with a patient, thereby maximizing the likelihood of success in the patient's treatment and/or management of the disease. Accordingly, the present invention further includes the ability, via the patient report card or similar means, for a professional staff person 170 to provide not only direct benefits (e.g., no-cost drug samples 174) and patient information, but also to provide opportunities for the patient to receive additional benefits (e.g., pharmaceutical coupons) and information.

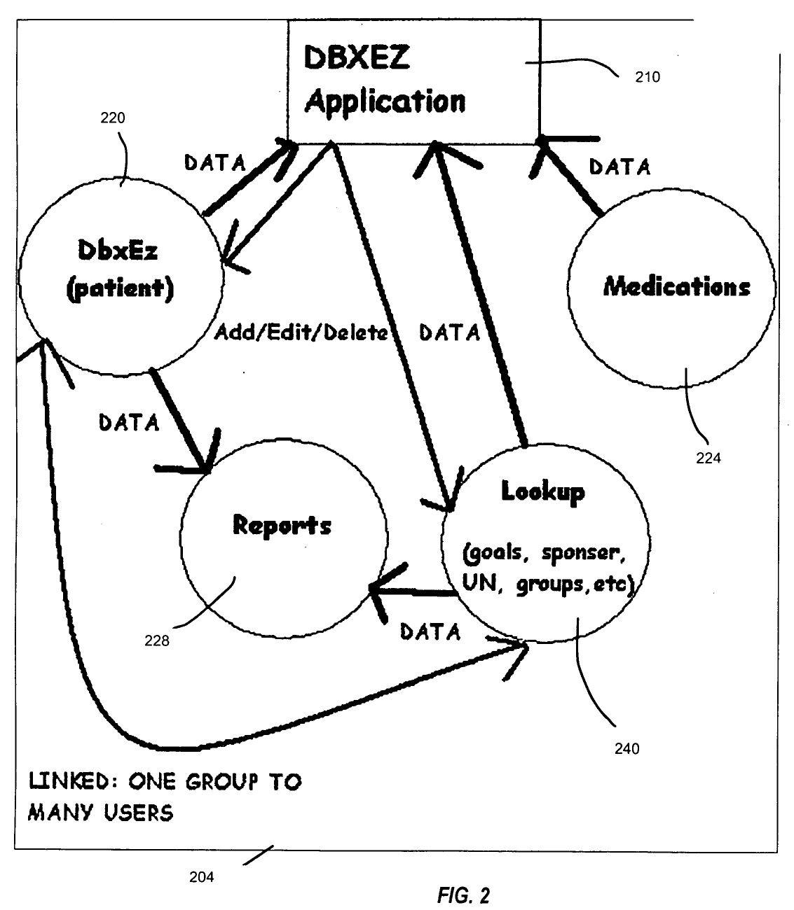

Referring also to FIG. 2, there is depicted a generalized data flow diagram for an embodiment of the present invention. In one embodiment, the method(s) of the present invention operate on a computer system to enable healthcare professionals to provide treatment to patients with a chronic disease according to a standard of care protocol. As represented by reference numeral 204, the present invention may operate within or in association with the larger architecture or framework of an electronic medical records (EMR) system 204. The embodiment employed as an example for the present invention is directed to the treatment of diabetes (DbxEZ™), although it will be appreciated that the present invention may be employed for various other diseases. As illustrated in FIG. 2, the application may be distributed in a self-installing application or agent 210 (other formats are possible, as is the possibility that aspects of the present invention are embodied with or associated with a medical records system 204), that operates on a Microsoft® Windows or similar operating environment. Three primary databases are implemented in accordance with the present invention, a patient database 220, a medications database 224, and a reports database 228. As illustrated, various items within the databases or lookups may be edited, and the databases are relational in that at least a group/user relationship is identified and recorded in the database.

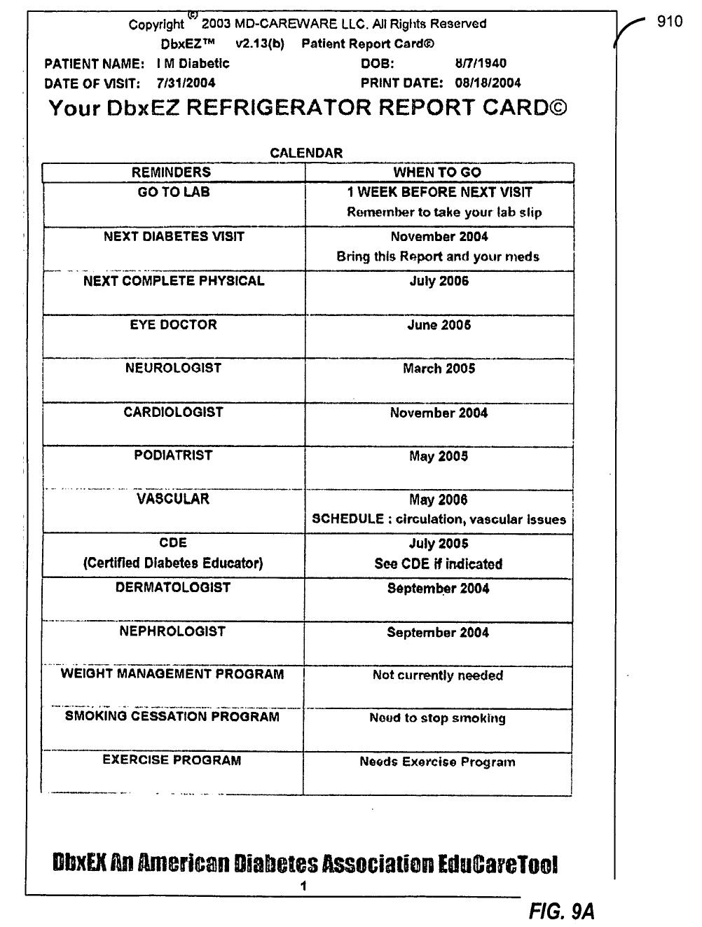

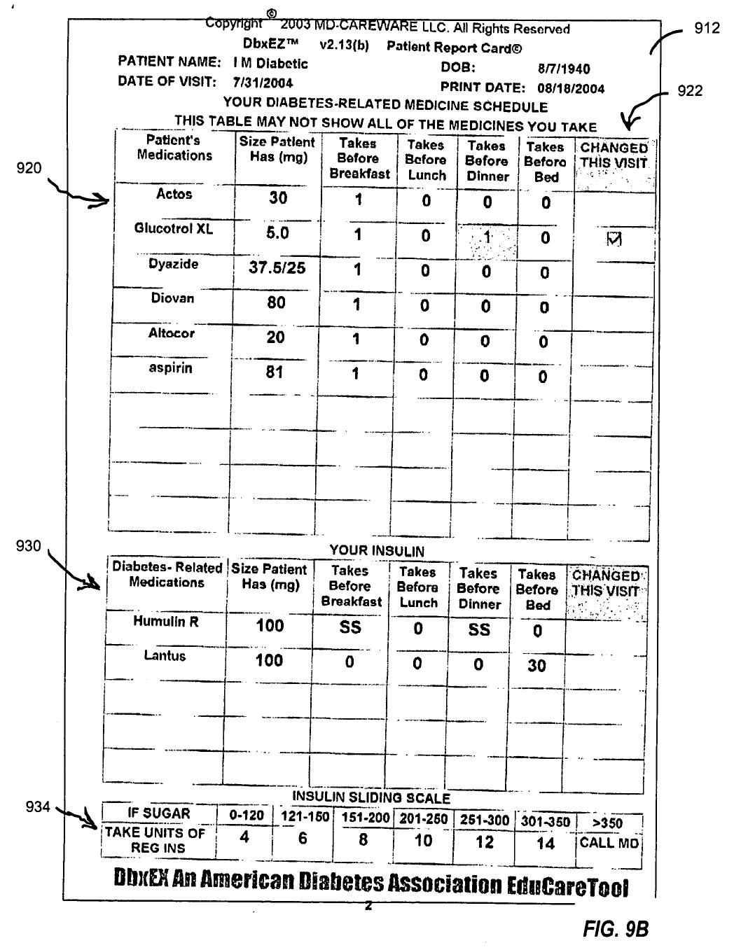

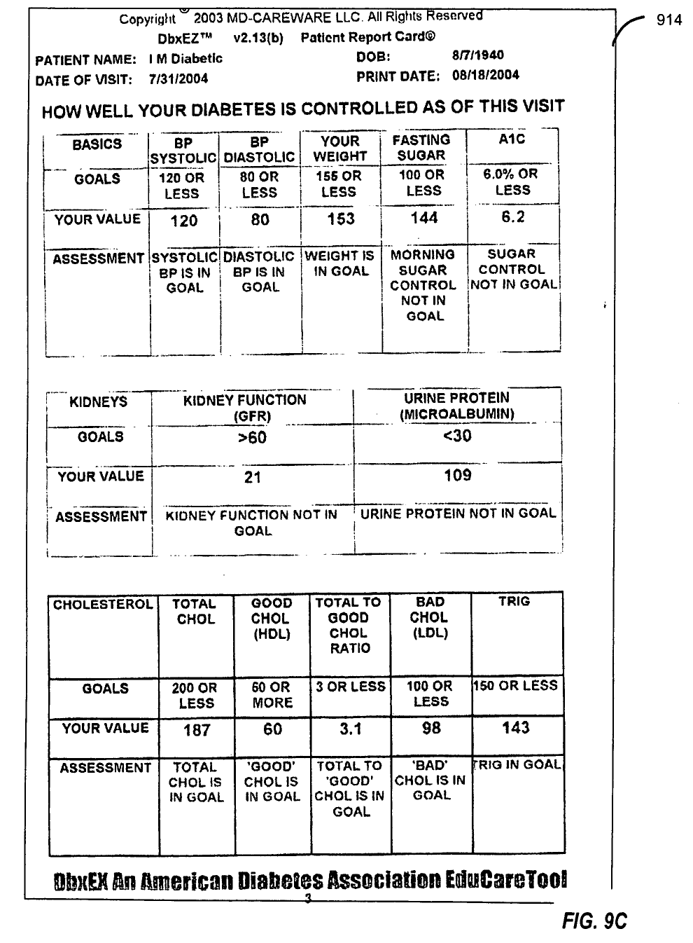

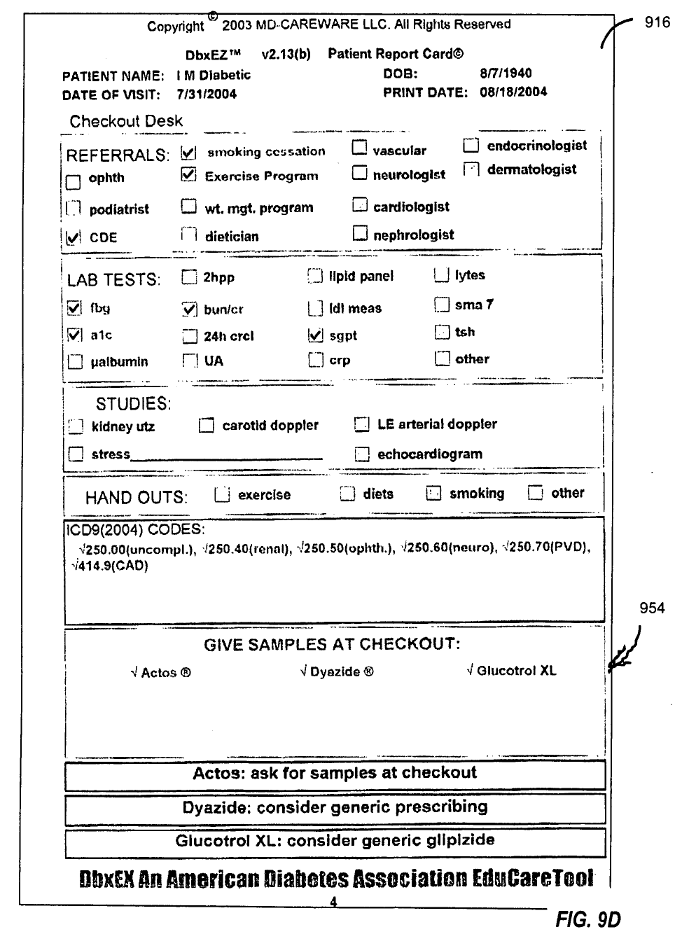

In general, the system operates in accordance with the pre-programmed application software operating on workstation 120 to enabling, in accordance with pre-programmed code in application 210, collection and entry of patient parameters associated with the standard of care protocol. These parameters are stored in the patient database 220 on storage medium 130 (FIG. 1). As noted above, the patient database records are used, in association with the medication and report databases to automatically generate a patient report card 154, the report card indicating at least information pertaining to the patient's parameters. Moreover, the report card may be automatically printed for the patient, and may include a disease-management calendar showing a future visit, and any lab dates, etc. as illustrated in FIG. 9A. The report card may also include a complete medication schedule as illustrated in FIG. 9B, wherein the schedule includes any changes to medication in accordance with the healthcare professional's recommendation.

The present invention consists of several functional units embodied in the application computer code 210. It will be appreciated that although one database arrangement is represented in the databases on CD-ROM Appendix, alternative coding may be utilized without departing from the intent and novelty of the present invention. The functional units of the present invention are: the Desktop; the Patient Diabetes Report Card; the Provider Chart Note; the PatientLink™; and various System Administration Functions.

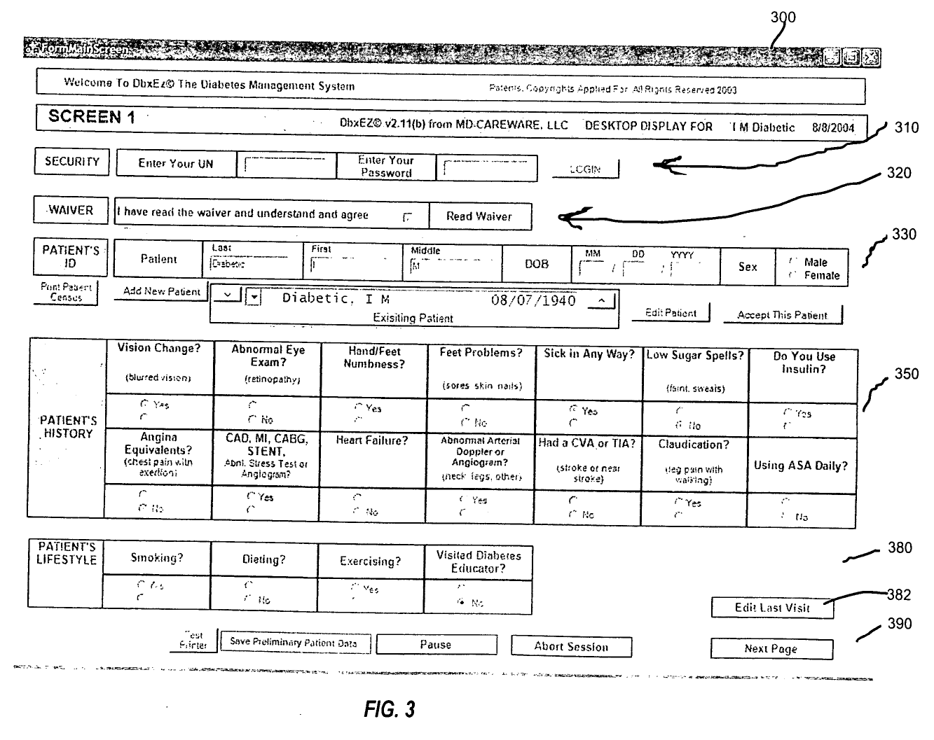

Referring to FIGS. 3-8B, the Desktop display is the result of the application software, which creates computer-display screens that are the portals of entry of patient disease information (patient parameters) by the healthcare provider. The screens sequentially flash and/or otherwise prompt the provider to enter, in an ordered way, the recommended data embodied in the appropriate standard of care. In one embodiment, entry opportunities cannot be avoided without such avoidance being recorded. All patient disease status determinations, treatments, and patient management decisions and conclusions are derived from entries made in the Desktop by the healthcare provider and subsequent processing by the application program.

In accordance with an aspect of the present invention, the Desktop embodies the patient-care parameter requirements of an authoritative body, such as, but not limited to, the American Diabetes Association, then translates the Standards of Care into a unique, inclusive, data entry format that permits entry of recommended variables and parameters for the patient. The data is recorded in the database and stored. The patient data entry process is steered using fixed sequences and flashing entry prompts to obtain patient demographics, history, medications, physical findings, laboratory values, consultant visits and more. It will be appreciated that entry of some patient data is required by such a system, whereas other data may be optionally entered. In one aspect the present invention provides a method to construct a patient calendar to track ADA recommended tests and referrals. The collected data provides the basis for all decision making and disease status determinations by the system in conjunction with the healthcare provider, and retains a “standard,” fixed format structure.

FIGS. 3-8B are representative screen shots of one embodiment of the Desktop Display. Each and all of the illustrated features depicted in the user-interface screens control and determine the information and the flow of information entered into system. FIGS. 3-8B are intended to represent an embodiment of the present invention, however, it will be appreciated that the embodiment contains features and functions that may be dependent upon or associated with a particular disease (e.g., diabetes) or a standard of care (e.g., ADA Standards of Care For Patients With Diabetes Mellitus). Accordingly, the representations in the Desktop embodiment depicted are not intended to be limiting. Other embodiments may include other parameters or exclude shown parameters, as the provider may decide or need. Other embodiments may also reflect other standards of care, or other diseases inclusively or exclusively.

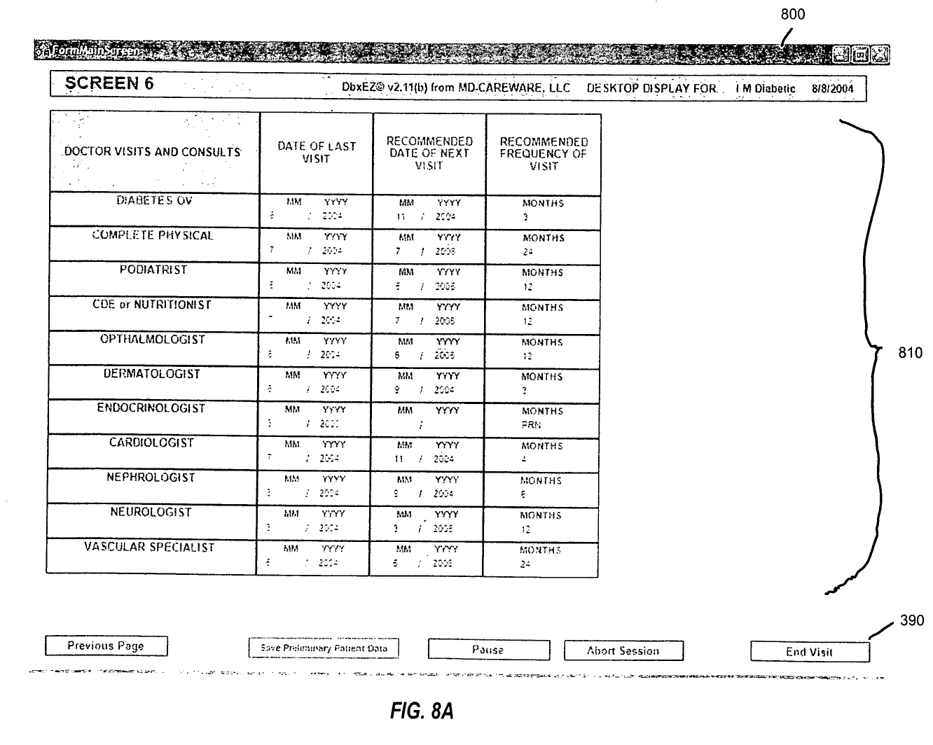

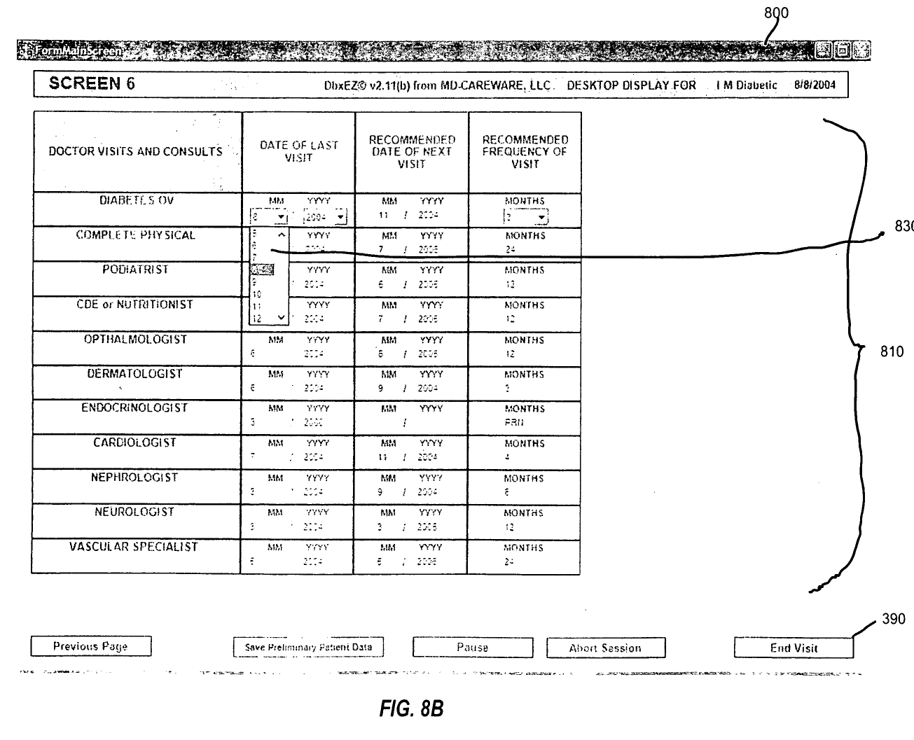

FIGS. 3-8B, for example, show one of the invention's embodiments reflecting ADA Standards of Care selected named parameters, including a displayed order of named parameters and patient parameter entry order, which is from left to right and top to bottom, screen one (FIG. 3) to screen five (FIGS. 8A-8B), or more or less screens as required. Other preferred embodiments may vary in wording and content. Each sequenced, ordered cell, in the depicted embodiment, prompts the user to address that cell in that sequence, for example by flashing that cell and requiring the information to be entered before advancing to the next cell or field. In another preferred embodiment, cells or fields may be skipped, and may be recorded by the system as being skipped.

Referring to FIG. 3, Screen 1 of the system is depicted and includes several features in window 300. In region 310, the security features of the present invention require a user name and password to permit access to the application and data (in addition to any such security of the workstation or its operating software (e.g., Windows®). Region 320 includes a patient waiver, where a patient can be prompted to read a disclosure relating to the use of their information and storage by the healthcare professional. Although the details of the waiver are not depicted, such waivers are known, particularly relative to HIPPA requirements. Region 350 includes various fields in which a user is prompted for data about the patient's history (either as new data or as changes to existing data). Accordingly, the present invention provides for entry of some or all of the items in a standard of care, and in a manner and form to allow and provide computer analysis of those items to determine the patient's disease state and management.

Each and every item shown in FIGS. 3-8B, including the items contained in any used standard of care, possesses a unique variable or field name. The variables are further represented in the attached Appendix, including listings of the associated databases, etc. and correspond to the input screens shown in part or full in the Figures. For The variables are an integral part of the embodiments depicted and allow the computer to analyze, calculate, and otherwise act on entered data by means of formulas, calculations, comparisons and other techniques that are carried out in accordance with pre-programmed instructions. It will be appreciated that through the use of a database, certain operations may be implemented via database query or reporting functions, whereas other aspects may be accomplished by application code. In the embodiment depicted and represented in the Appendix, the database is a Microsoft® Access™ database with Visual Basic interface (or series of databases as depicted in FIG. 2), and operates on a .NET framework (Version 1.1), although other databases and software systems may be employed to achieve similar functionality.

More specifically, region 330 seeks input relating to the patient's medical history, since the last visit, as Vision Change? (blurred vision), Angina Equivalents? (chest pain with exertion), Claudication? (leg pain with walking), Hand/Feet Numbness?, Feet Problems? (sores, numbness), Low Sugar Spells? (faint, sweats), Infections?, Heart Failure?, (class III, IV). Region 380 of Screen 1 allows the user to input data pertaining to the patient's lifestyle or changes since the last visit, such as Smoking, Dieting, Exercising, Using ASA Daily, Visited Diabetes Educator, Insulin Use, etc. Button 382 also allows a user to edit information stored in a previous visit in the event that an item was mis-characterized. Region 390 is for navigation through and interaction with the application and associated data entry screens. The buttons depicted in region 390 are self-explanatory.

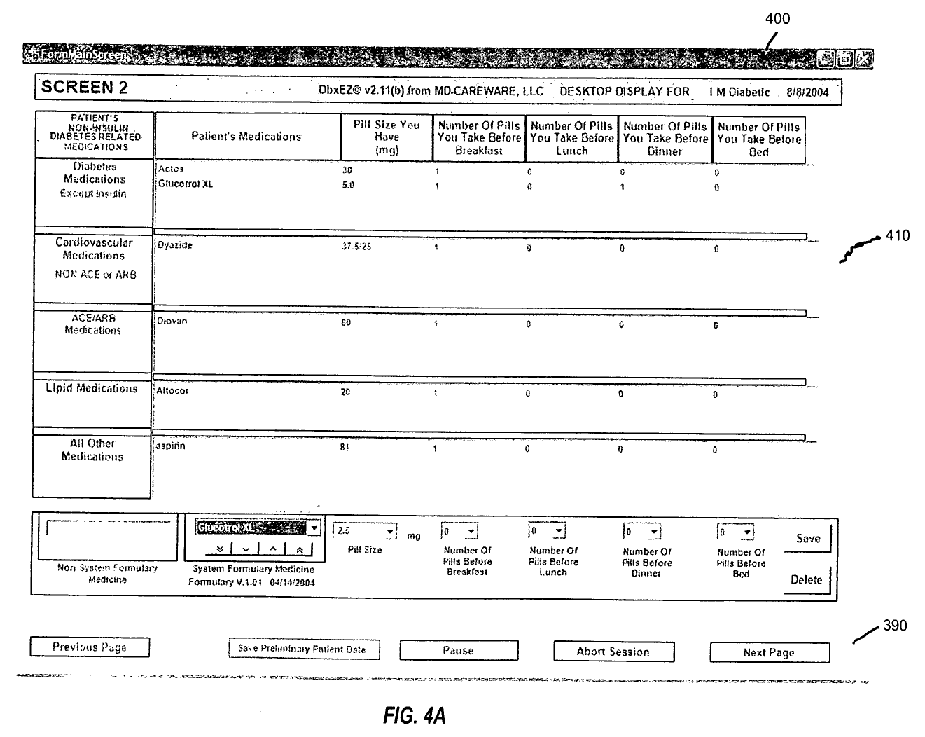

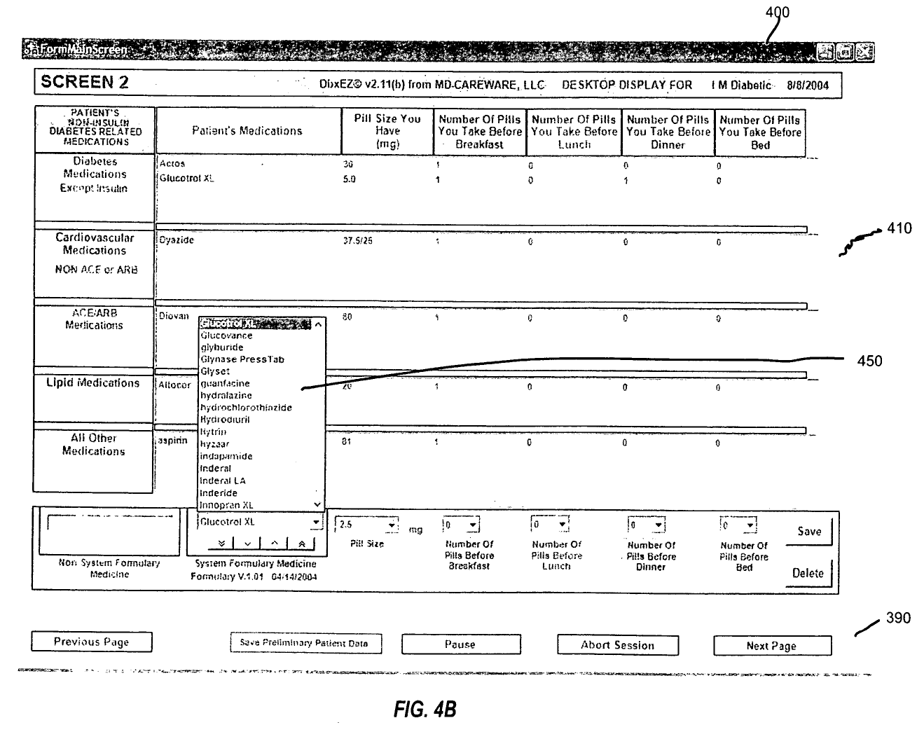

FIGS. 4A-4B, further to above, show a second display screen-Screen 2 in window 400. In this screen, and the layout depicted, the user enters data in relation to the patient's medication(s) and other selected parameters and wording to provide responses or answers indicative of the patient's medication use. Further, this screen allows the user to scroll through the patient's medications stored in the computer's formulary to edit the entry, delete medications or add new medications. This feature allows the computer to ‘know’ what medications the patient is on in order to analyze interactions, contraindications, and similar and to ‘link’ the patient to medication samples provided by manufacturers using another feature of the system referred to as PatientLink™. The availability of medication samples is indicated through one of several ways, including entry by a user, uploading from a network (e.g., data indicating samples inventory, sample inventory updates from manufacturer, etc.), or equivalent means for entering sample availability information. The availability of the sample medications (or other prescriptive samples) may be separate from the formulary medicine data that the system relies on as possible medications to employ with the disease. It is during the processing of a patient's data, based upon entered information and the healthcare providers diagnosis and selected treatment regimen, in which a prescribed medication is “matched” with samples available in the provider's inventory in order to determine if the patient would receive a sample (e.g., as indicated on the patient report card in the PatientLink™ section (described below). In region 410, the user is prompted to enter the patient's non-insulin diabetes related medications. As illustrated, column headings for this region include: medication name, pill size patient has, pills you take before breakfast, pills you take before dinner, pills you take before bed, etc. As illustrated in the pop-up menu 450 of FIG. 4B, the various medications may be pre-programmed and thereby selected by the user of the system. Similar pop-up, or pull-down menu selection functionality may be provided for other entries or fields to facilitate data entry. As further observed within region 410, the row headings include: diabetes medications, cardiovascular or blood pressure (BP) medications NON-ACE/ARB, ACE/ARB medications, lipid medications, and other medications such as Aspirin.

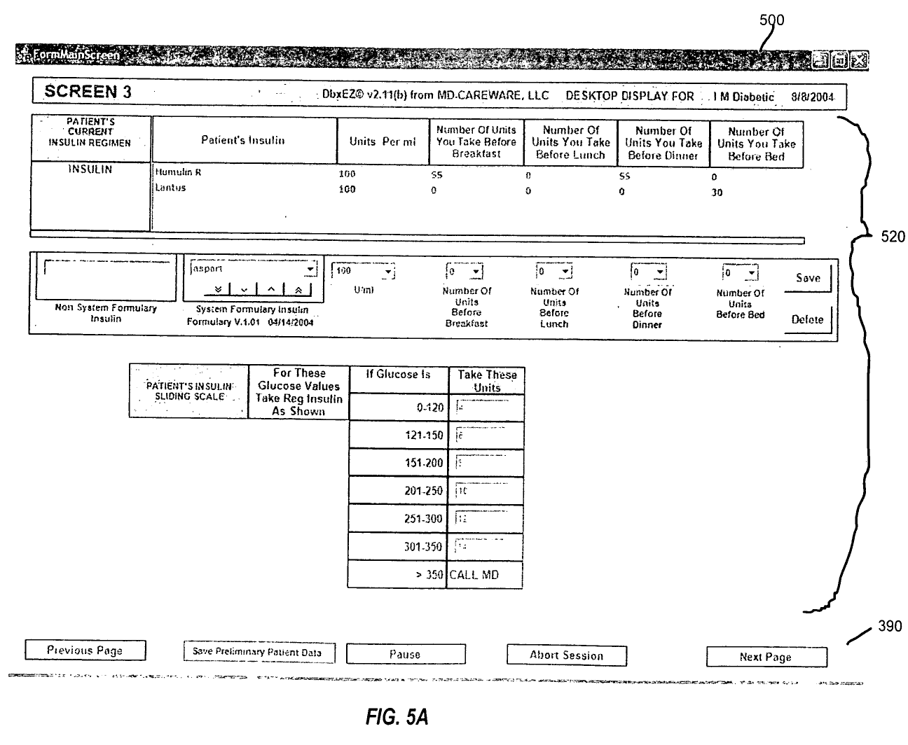

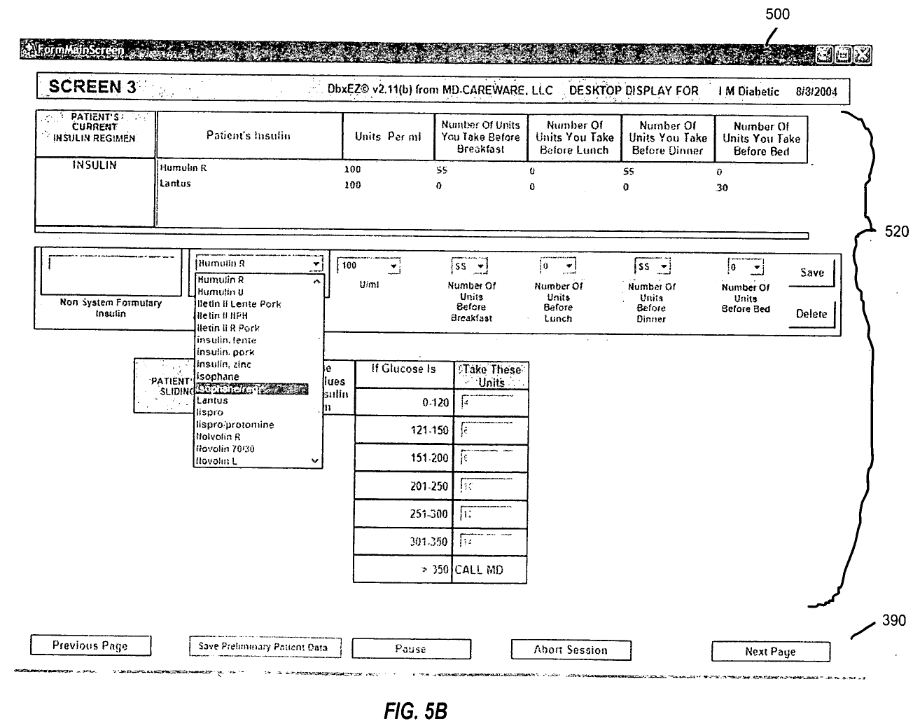

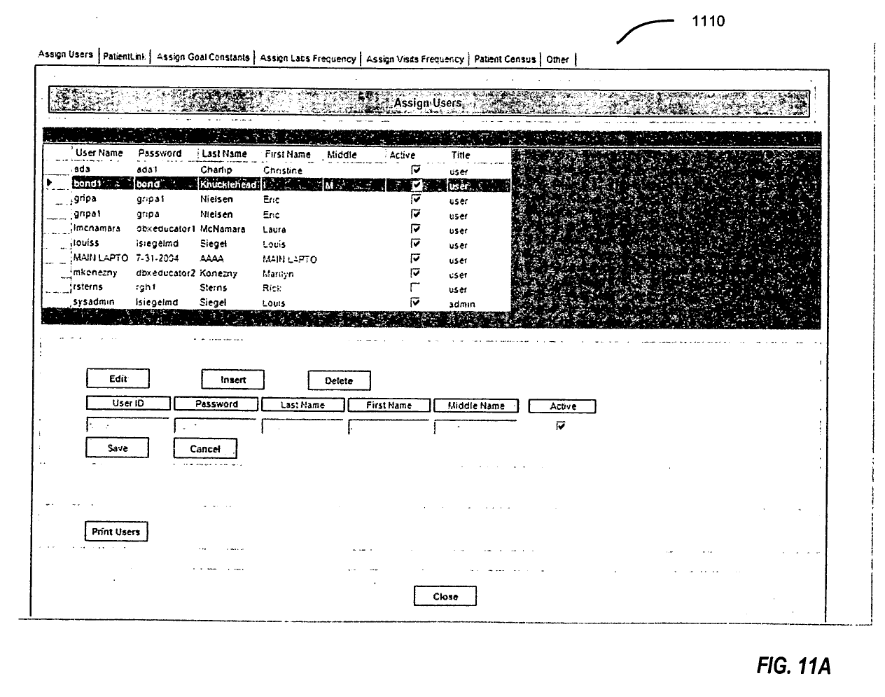

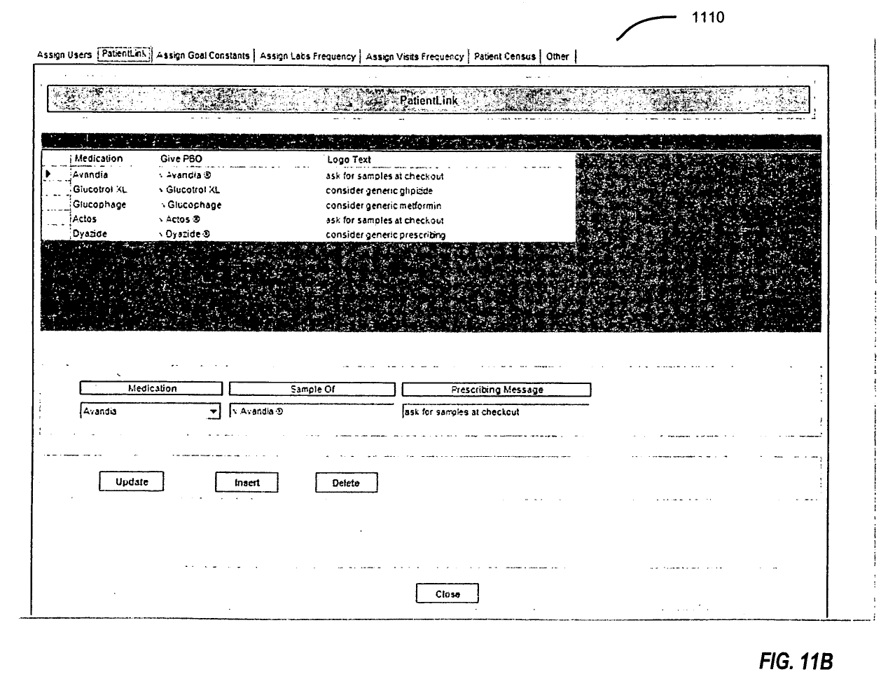

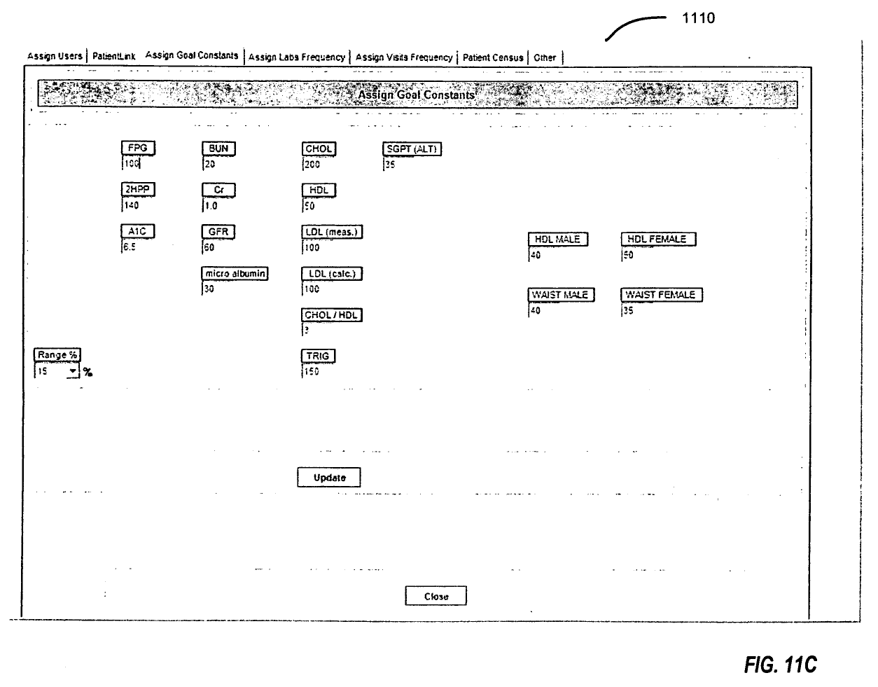

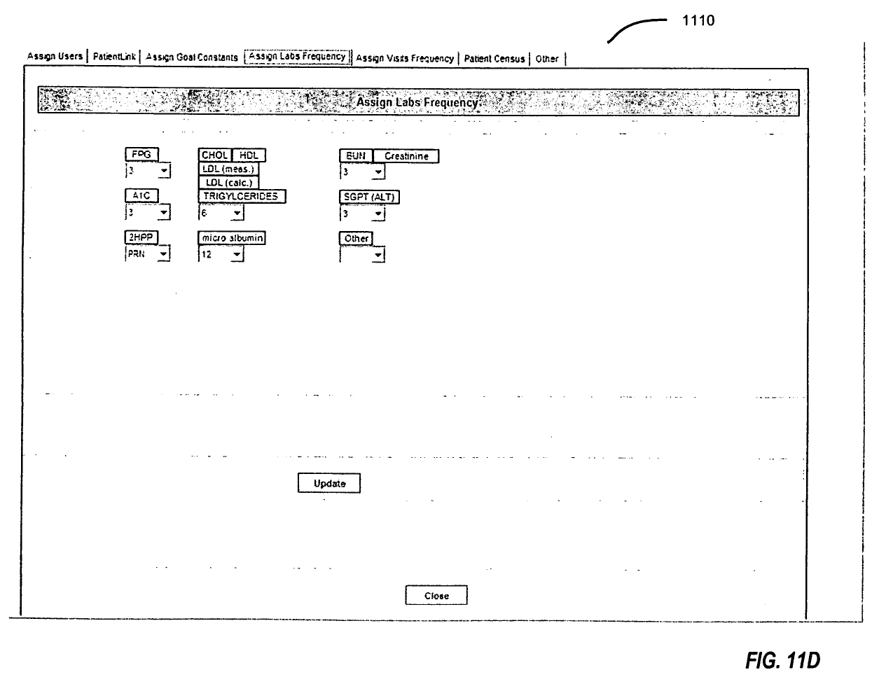

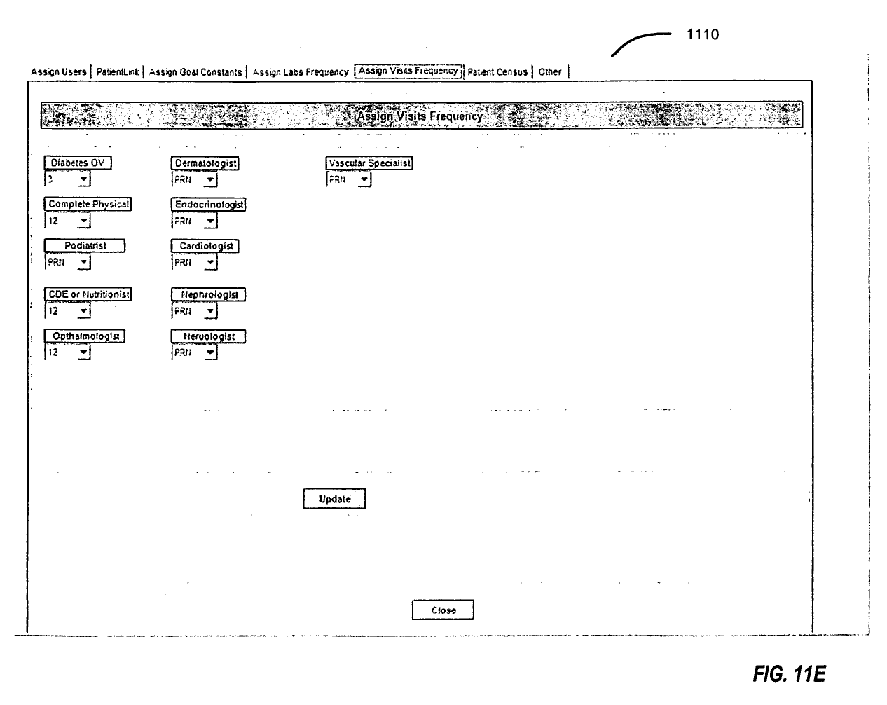

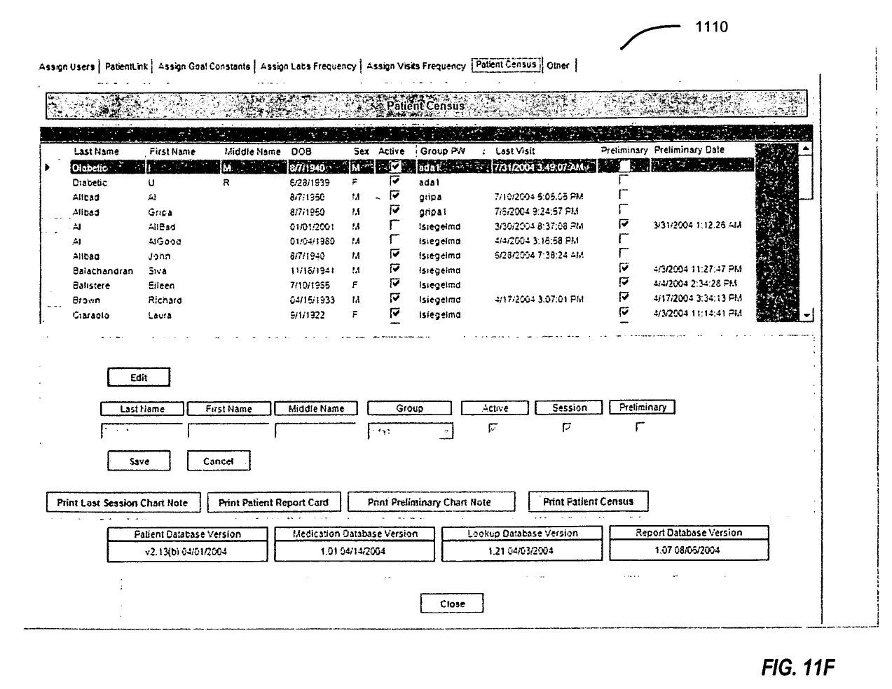

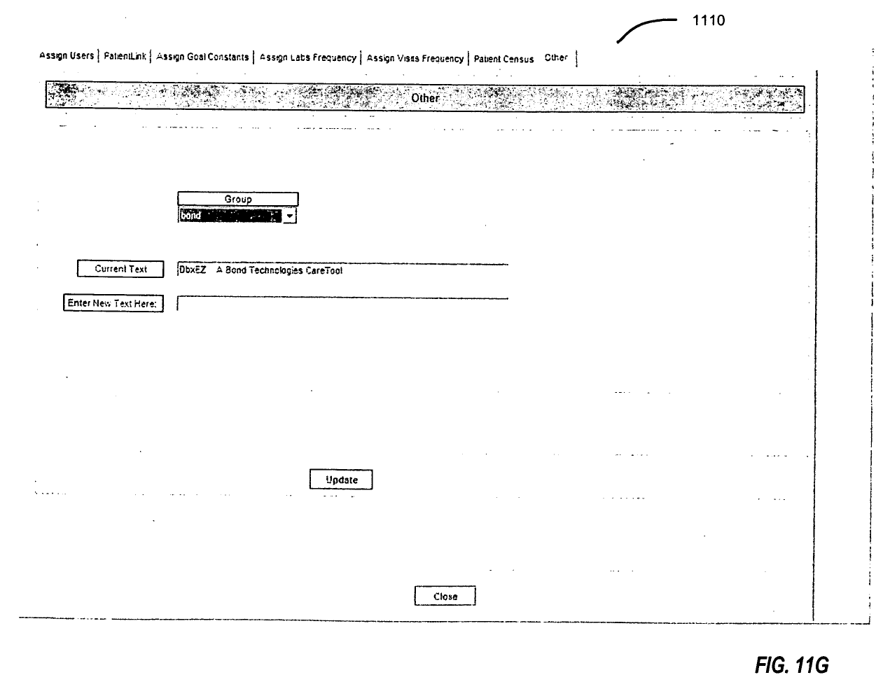

Referring next to FIGS. 5A and 5B, depicted therein is Screen 3, similar to Screen 2, but where a user enters the diabetes patient's current insulin regimen in region 520. In region 520, the data entered includes type of insulin, as well as the amounts taken at various times during the day (e.g., before breakfast, before lunch, before dinner, before bed). Again, as depicted in Figure B, the various entries may be streamlined through the use of pre-specified entries depicted in a pop-up or pull-down menu. As will be described in detail with respect to FIGS. 11A -11G, the various entries may be edited using the administrative functions of the present invention. The rows accommodate various types, including, for example, Regular, NPH, and Lantus, while the columns seek information relative to the amounts. In the bottom of region 520, the Patient's Insulin Sliding Scale is depicted, allowing for entry of specific data dependent upon a glucose level.

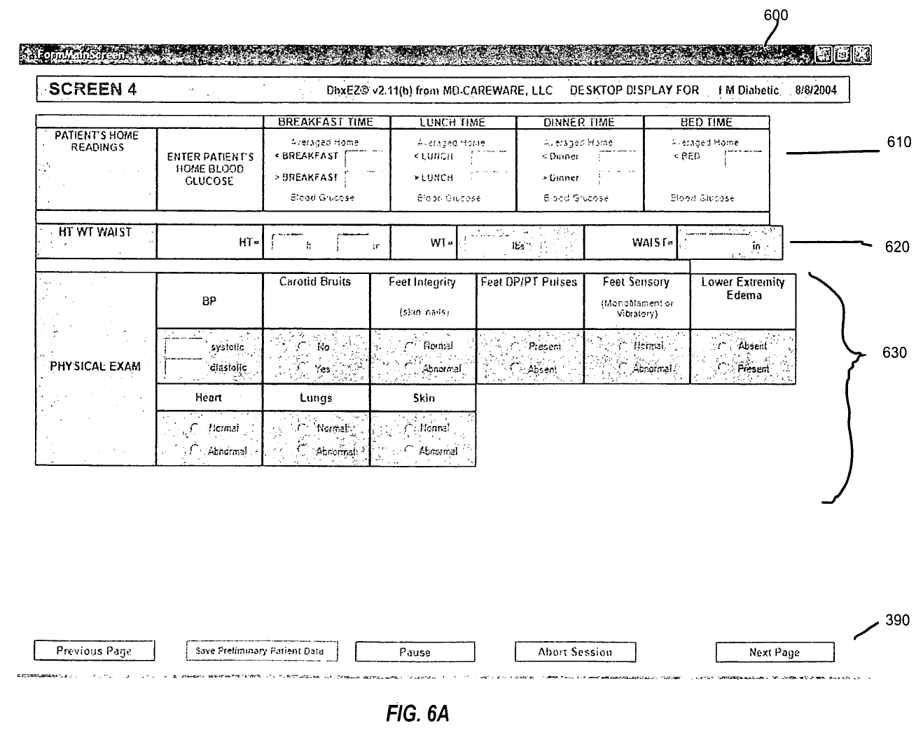

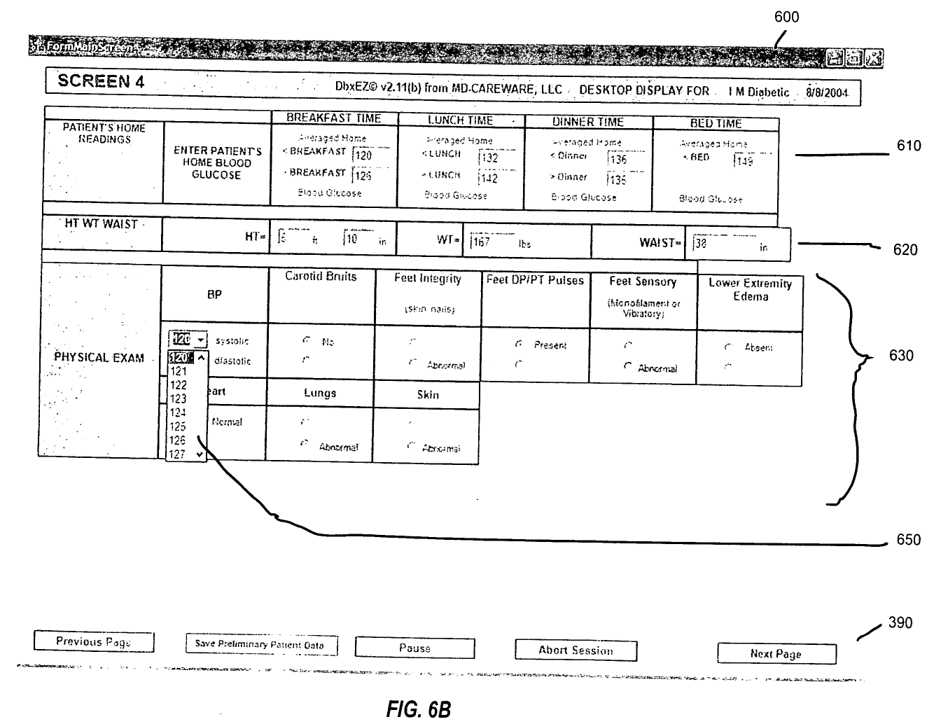

Referring also to FIGS. 6A and 6B, Screen 4, as depicted in window 600, shows one embodiment of the computer display screen providing the user the specific format, layout, selected parameters and wording and the place to enter responses related to the patient's home glucose readings (region 610), as well as measurements such as height, weight, waist (region 620), and physical examination results (region 630). As will be appreciated, and as described above, the various fields or cells may be completed by keyboard entry, stylus selections and/or menus (e.g., 650 in FIG. 6B), or other data entry means.

As illustrated by the figures, Screen 4 seeks, responses or data relating to the following: patient's home readings, fasting or home blood glucose (FBG), before/after breakfast, lunch, dinner, etc. and averages of one or more. Region 620 seeks the patient's data pertaining to physical measurements for height, waist in inches, and for weight in pounds (lbs.).

As shown in region 630, the physical examination section seeks patient parameter data that would be the result of a physical examination by a healthcare provider. Although various forms of data may be included, the embodiment depicted seeks at least the following: blood pressure (BP) for systolic and diastolic, Carotid, Bruits, Feet Integrity, Feet DP/PT Pulses, Feet Sensory (Monofilament or Vibratory), etc. Each of the selected entries requires a data value or an indication as to whether the item is observed to be present or absent, or normal/abnormal, etc. as appropriate for the item being observed or measured.

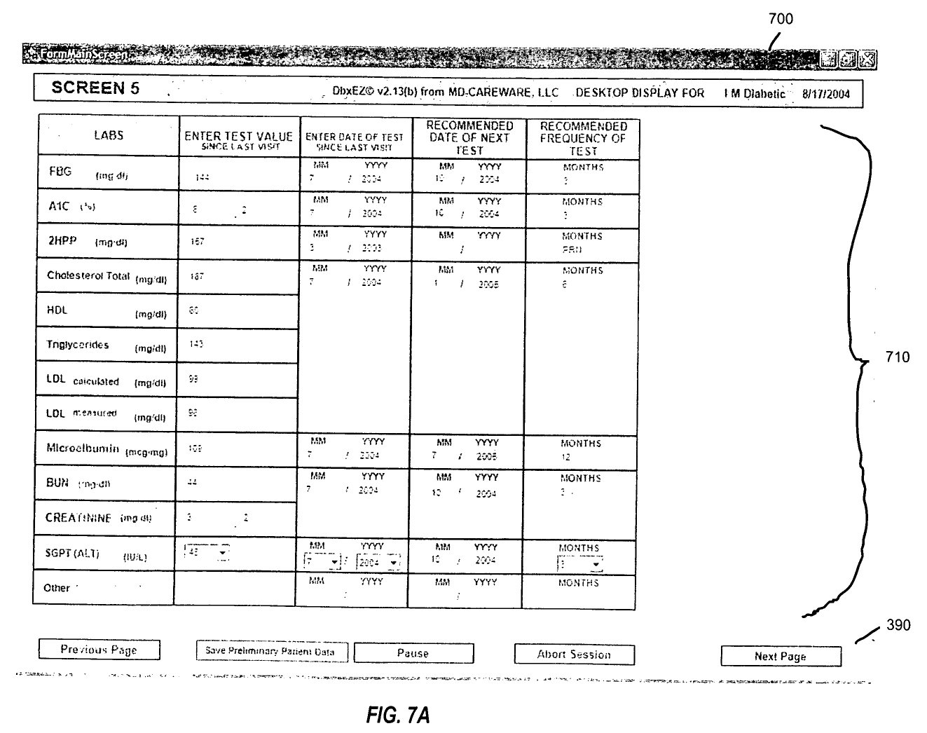

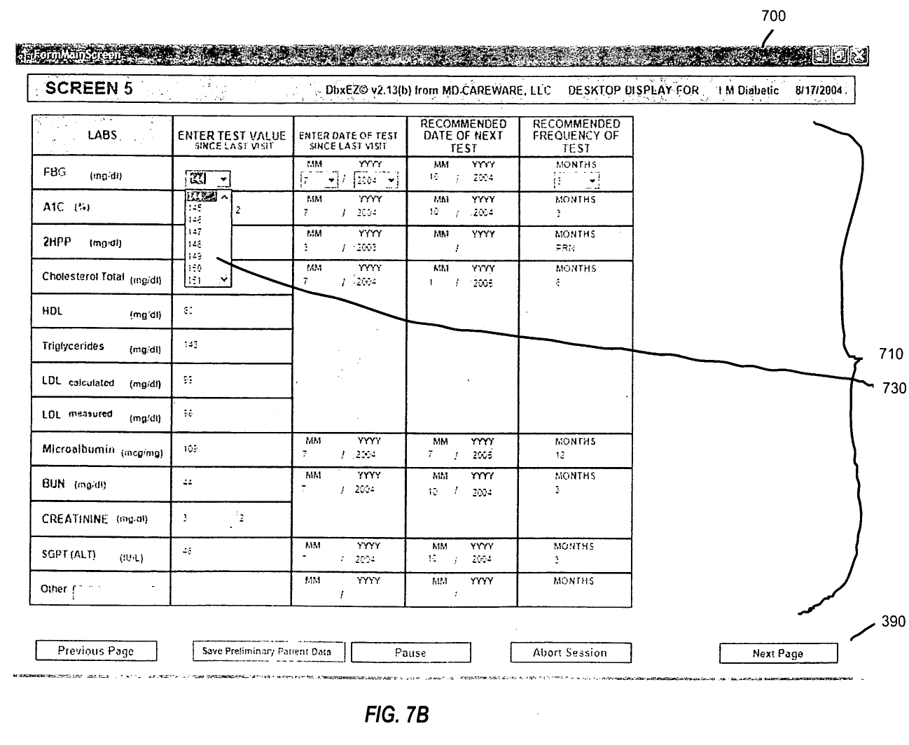

FIGS. 7A and 7B, further to the above, show display Screen 5 in window 700, providing the user a place to enter responses or answers to questions related to the patient's pertinent laboratory test results/values and for the computer to display previously entered or calculated test dates and test frequencies. The entries, as are all entries into the Desktop screens, are used as parameters to analyze the patient's disease state and make recommendations related to diagnosis, treatment, management and other pertinent aspects of the patient's care. The Date of Last Test and Recommended Frequencies fields enable the user, via the system and pre-defined rules or guidelines set forth in the standard of care, to monitor patient testing compliance. Again, the default or pre-defined frequencies and other criteria for patient evaluation may be adjusted by access to the administrative screens as described below. Similarly, as depicted in FIG. 7B, a pull-down menu 730 may be employed in order to provide a user with a plurality of responses for selection.

Referring next to FIGS. 8A and 8B, there is shown a display screen, Screen 6, which displays and interactively prompts the user with a place to enter by keyboard, stylus, or other means, responses or answers to questions related to the patient's pertinent doctor visit history and for the system to display previously entered or calculated visit dates and visit frequencies. The entries, as are all entries into the Desktop interface screens, may be used as parameters to analyze the patient's disease state and make recommendations related to diagnosis, treatment, management and other pertinent aspects of the patient's care. The date of last visit and recommended visit frequencies, by pre-programmed defaults, enable the user and system to monitor patient primary care physician and consult visit compliance.

In region 810, the system prompts the user to input doctor visits, date of last visit, recommended date of next visit, recommended frequency of visit, and in each field therein seeks a date. Moreover, as illustrated in region 830, the present invention seeks such visit information relative to various healthcare providers, including primary care physician, podiatrist, CDE or nutritionist, ophthalmologist, dermatologist, endocrinologist, etc., with corresponding dates and frequencies for each. Again, entry of information is facilitated by the use of pull-down menu 830, to facilitate non-keyboard entry of data, or to suggest data that may be entered. It will be further appreciated that various features of the computer operating system (e.g. Microsoft® Windows) may provide additional fill-in functionality (e.g., automated entry, proposed response based upon typing of beginning letters, etc.).

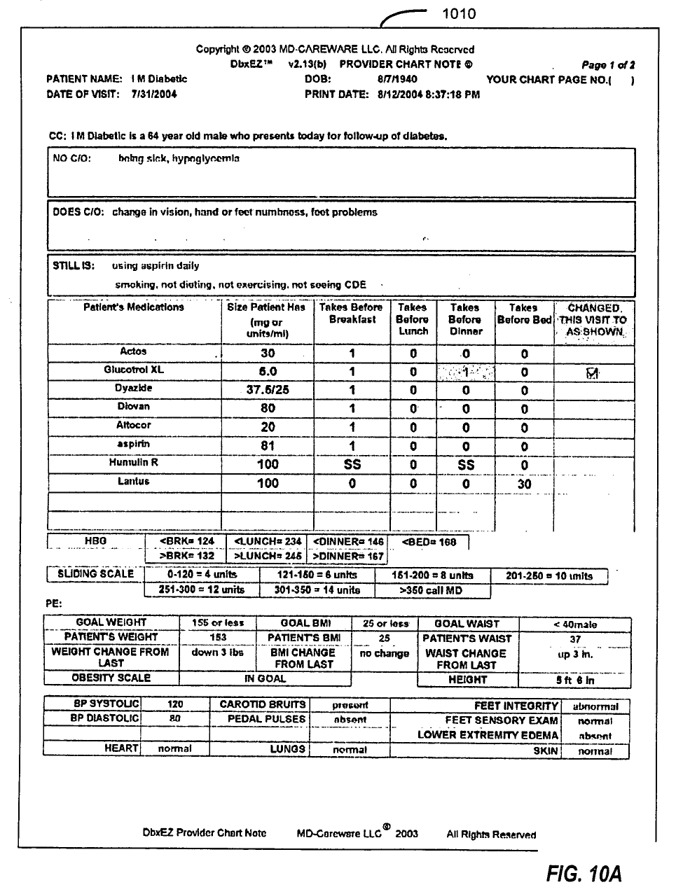

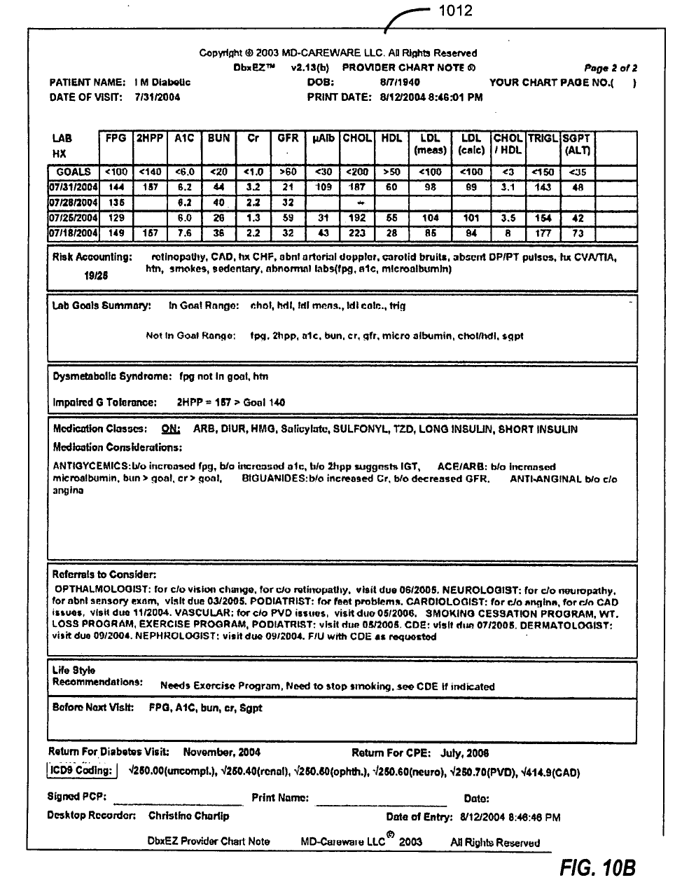

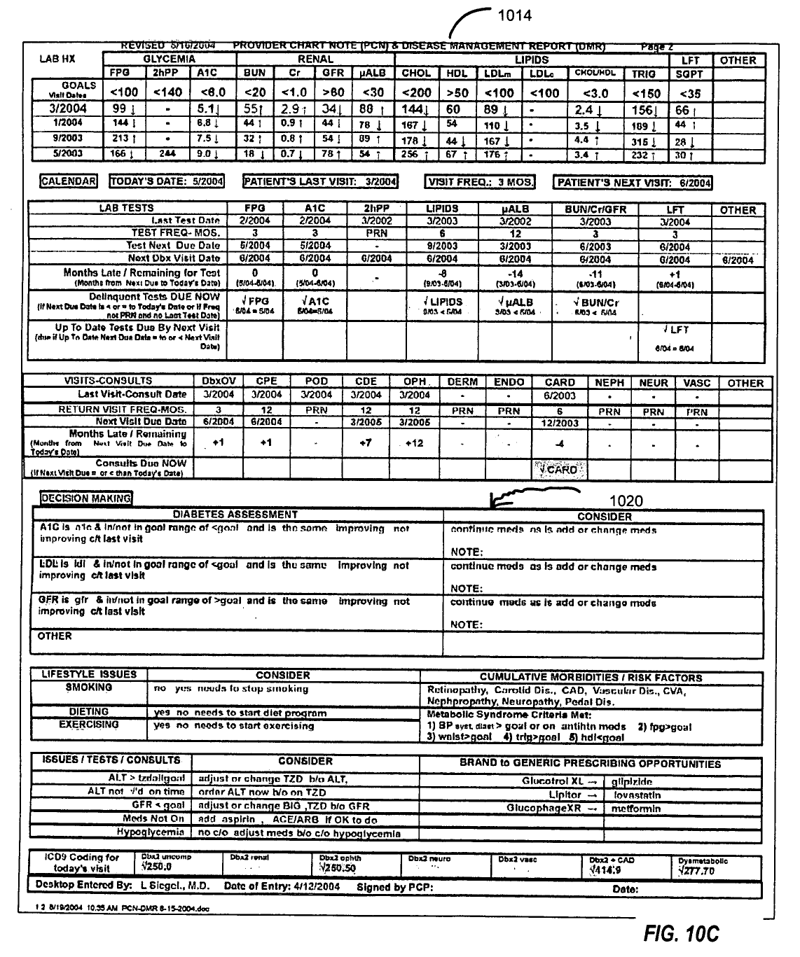

At various stages during the entry of patient data, and at least upon completion of entries on one or more of the six screens previously described, the system operates to generate one or more pre-defined “reports” and in which the system prepares a formatted output such as the patient report card (FIGS. 9A-9D) and provider chart note (FIGS. 10A-10C). It will be appreciated that certain calculations, comparisons and similar functions, including logical processing of results must be carried out in order to process the data and provide the reports. For example, the data relative to glucose levels in the present example must be compared to desired ranges for the patient and a report might indicate whether the result is within, near or out of range. Having described the input of information into the system, attention is now turned to the exemplary reports described above.

FIGS. 9A-9D show an embodiment of one report that is automatically generated by the system, called the Patient Diabetes Report Card™. Page 1 of the report card (910) indicates that the printout is intended to be provided for the patient to display on a refrigerator or similar prominent place in the home so that the patient can refer to it. Page 1 includes the patient's info at the top and then provides a calendar or tabularized representation of the future office visits, tests, medical consultations, etc. as were recorded or recommended by the healthcare provider and recorded in the database. Accordingly, this novel report is automatically generated by the software based on entries made into the DbxEZ Desktop, and is printed for review with the patient.

Page 2 of the patient report card (912) is illustrated in FIG. 9B. Page 2 includes a summary of the patient's recommended medications, with the upper table 920 indicating diabetes-related, non-insulin medications, including the frequency, timing and dosage of each, as well as a column 922 that highlights any medications that were changed in the current office visit. The middle table 930 is an indication of the patient's insulin, again indicating the timing, dosage, etc. The lower table 934 is a sliding scale for insulin dosage that has been tailored for the patient, and is representative of data input into Screen on FIGS. 5a and 5B.

The report card, particularly at page 3 as illustrated in FIG. 9C (914) also indicates a status of the patient's management of the disease. In the three tables illustrated, the system prepares summaries of the patient data and includes a comparison of the data by indicating via color, or in alternative display means (e.g., sliding scales, graphs, etc.), whether the parameters are within or near a desired range (green), or out of range (red). It will be appreciated that additional indicators may be used to represent status versus goals, and that finer resolution may be employed. It should also be understood that the ranges may be adjusted as well as illustrated in FIG. 11C. For example, in addition to specifying range values, it may be that the system user specifies a target value and then selects a range (percentage) about the target that would be acceptable. Simple logic may be used to determine if a particular reading is in or out of range. However, the present invention may further employ fuzzy logic to determine if the actual reading is near the range, based upon, for example, the range size. Thus a large range might have a narrower “near range” set of values whereas a small range might have a larger “near range” set of values.

Referring also to FIG. 9D, the last page of the report card (916) provides information to the patient for use upon checking out of the healthcare provider's office. In addition to indications for referrals and lab tests to be scheduled, page 916 includes region 950 to indicate information to be provided to the patient and region 954 to indicate what medication samples are to be provided. Here again, the output in region 954 is automatically generated as a function of the provider's prescribed medication as well as the availability (inventory) of the association medication samples. Region 954 may also include, although not specifically depicted (see FIG. 1), coupon boxes at the bottom thereof to encourage patient's to redeem the coupons for medications or other recommended items (testing kit supplies, etc.).

Region 954 of FIG. 9D is referred to in the DbxEZ™ system as the PatientLink™, and includes the printing of the samples to be dispensed or other offers by manufacturers of the medications/supplies the patient is on—here, Actos® and Glucophage®. The PatientLink feature is shown in the present embodiment as an integrated feature of the report card. However, it will be appreciated that the PatientLink functionality may be implemented as a stand-alone feature or as a feature within another system such as an EMR system, that informs, delivers, or otherwise provides to patients, in a professional and ethical manner, what may be called pharmaceutical manufacturer's patient-benefit opportunities. Such opportunities may include drug samples, other supplies, sample offers, rebates, discounts, vouchers, educational and support services, and more. In the present invention these opportunities are prepared, on a periodic basis, by one or more manufacturers or marketers of diabetes medications or supplies. The patient benefit opportunities exist in two forms, real and virtual. The ‘real’ form is the actual material eventually received by the healthcare provider's office and given to the patient. The ‘virtual’ form is the computer coded form provided by the manufacturer to DbxEZ software and delivered, periodically, to the software via CD, network (e.g., Internet) other means. The feature may also match branded drugs to available generics for cost-saving prescribing.

As described above, the system ‘knows’ what medications a patient is on by entries made in the Desktop and the software ‘knows’ what the benefits (samples) are when the ‘virtual form’ is included. In this way, the benefits are automatically flagged and provided in a variety of forms, if approved, to the patient at the time of the office visit. The process is controlled by routines that link manufacturer's benefit packages, uploaded to DbxEZ, to drugs or other medications the patient needs or may be determined to be beneficial. The system matches the patient's medications to the benefit packages and then prints all relevant benefit opportunities on the patient report card. The invention may also provide for the patient to designate that such benefits are requested and for the physician or provider to approve, so that the system automatically makes the benefits available to the patient at the checkout window or other site. Specifically, the report card includes a unique ‘checkout’ menu (954) that includes the linkage of the patient to needed and available samples or other benefits. Hence, the system (PatientLink™) provides a method and software that professionally, HIPPA compliantly, and ethically links providers, and patients to pharmaceutical manufacturers for the purpose of delivering benefits to the patient. In this way, the PatientLink feature facilitates the awareness and delivery of patient cost-saving benefits for medications and supplies the patient already takes, or may need and want. The feature further provides an alternative to currently expensive, inefficient and in some facilities, not permitted, sampling programs, and couples manufacturers sampling efforts to a medically sound program that elevates the level of care of patients with diabetes. In summary, the PatientLink feature links, connects, and/or interacts with: (a) the pharmaceutical manufacturer and their prepared patient benefit packages, (b) the DbxEZ software or other medical software system, (c) the patient, and (d) the healthcare provider, all at the time of the office visit when the feature can be most beneficial.

Use of the patient report card, and the associated items described above is believed to improve the provider's and patient's communication and to facilitate management of the of chronic disease. As described, the present invention thereby provides not only the data entry and storage of patient parameters, but facilitates communication of the information discussed in an office visit, as well as assisting the patient in receiving benefits that may be made available from the provider and/or the pharmacists or pharmaceutical manufacturer. The entries into the Desktop, as shown in FIGS. 3-8B are, as described above, used as parameters to analyze the patient's disease state and make recommendations related to diagnosis, treatment, management and other pertinent aspects of the patient's care. The results of these analyses are displayed and printed in the form of the patient report card as illustrated in FIGS. 9A-9D.

FIGS. 10A-10B represent another report that may be generated by the system, and FIG. 10C depicts an alternative format for the representation of FIG. 10B. Referring to FIGS. 10A-10C, there are depicted a series of report pages 1010-1014. Each of the figures represents a page of a provider chart note that may be printed and stored in a patient's hard copy file for a permanent record. It will also be appreciated that the data remains stored in the system, and that an electronic copy of the report can be generated and stored on a network drive or similar storage medium for later access.

The Provider Chart Note™ reconstructs, analyzes and otherwise transforms the information contained in, or implied by, the Desktop software described relative to FIGS. 3-8B into a form physicians understand. The layout, format, content, coloration, style, and purpose of the provider chart note are directed to the healthcare provider and facilitate ease of access to patient data. This report, also generated automatically by the software logic, relies upon entries into the Desktop. The provider chart note, in one embodiment, contains the following items:
Date of Visit;
Demographics;
Chief Complaint as CC;
History as HX, with Complaints in the form, NO C/O and DOES C/O;
Habits;
Medications, as MEDS, derived from the Desktop formulary patient entries;
Physical Exam as WT, GOAL WT, WT CHANGE FROM LAST, BMI, GOAL BMI, BMI CHANGE FROM LAST, WAIST, GOAL WAIST, WAIST CHANGE FROM LAST, HT, BP SYSTOLIC, BP DIASTOLIC, CAROTID BRUITS, HEART, LUNGS, FEET Skin Integrity, Pedal Pulses, Sensory, etc.;
Additional Findings;
Laboratory History;
Goals Summary;
Referrals & Tests for consideration;
Lifestyle Recommendations made; and
Patient's Dx, which may further include by criteria, Metabolic Syndrome: by criteria, Goal Analysis: by criteria, Consults To Consider, by criteria, Medication Additions: consider, by criteria, Medication Deletions: consider, by criteria, Medication Deletions: consider, by criteria, Medication Changes: consider, by criteria, Tests To Be Done:, by criteria, Return Visit with: Dr for f/u dbx: date by criteria, CPE: date by criteria, Life Style Recommendations:, by criteria, Signature, by provider, MD'S NAME, DATE.

The summary sections include a novel collection of statements consisting of fixed text and text generated by the software based on criteria as described previously relating to conclusions, calculations, software decision logic and other methods embedded in the DbxEZ logic, as derived from data entered into the Desktop, to inform the provider of the essential disease state of the patient as it relates to the selected standard of care and the preferences of the provider, all of which is programmable into the system as briefly described below. As further illustrated at FIG. 10C, in the alternative chart note 1014, the chart may provide a decision making section 1020 that provides the healthcare provider with an assessment of particular results/parameters and automatically proposes treatments and alternatives for consideration. These treatments would be in accordance with a rule embedded within the system, which may be based upon a standard of care or similar recommendation.

Having described the basic operation of the system, attention is now turned briefly to the set-up and administration of the system. As previously described the system may operate on a workstation or as part of a networked system. The system is, however, flexible in that it may be modified in accordance with the preferences of a user, a healthcare provider's practice, etc. Referring to FIGS. 11A -11G, there are depicted various user-interface screens associated with the maintenance or administration of the system. Accessed by selection of the tabs at the tops of the windows 1110, the various features will now be briefly described.

FIG. 11A illustrates a user-administration feature, where user access to the system may be established. As will be appreciated, the data is entered for a user (or edited) in order to enable/control access to the databases. As depicted in FIG. 2, the user data is stored in one of a series of tables referred to as “Lookup” for access by the system. Referring to FIG. 11B, the PatientLink tab presents a user with a table indicating the various medications available via the PatientLink feature described above. In addition to the manual entry of information in this table (indicating what samples/benefits may be available), it is contemplated that such information may be uploaded from an alternate source, and that the benefits may include not only a “real” sample, but also the possibility of a coupon or similar ‘virtual’ benefit. It will be further appreciated that the PatientLink table, or an associated table, may include additional information, counters, inventory tracking etc. for inventory and/or regulatory controls.

Turning next to FIG. 11C, the Assign Goal Constants page provides a means for a user or administrator to set or edit goals for various tests or other disease parameters. These levels may be set on a global basis, and may then be modified for a particular patient. Similarly, the Goals tab provides access to the range field 1120. Field 1120 sets the default range within which a patient will be targeted for each of the various goal constants. In use, as noted above, the range is a percentage based upon the particular goal and defines the boundary between near goal or out of range which the system uses to automatically determine the patient's status on a particular goal.

Referring to FIGS. 11D and 11E, the Labs Frequency and Visits Frequency windows provides default values for the system to employ when setting upcoming visit of laboratory testing recommendations. As will be appreciated in light of the above description, the frequency settings are both for testing and visits with not only the primary provider, but for other medical consultations as well. The frequency settings may also be used to identify when a patient has exceeded a recommended period for visits, testing, etc. so that a report (provider note and/or report card) may indicate the same.

Considering FIG. 11F, the Patient Census tab in window 1110 reveals access to one of the tables in the patient database, where the basic information for a patient may be reviewed or edited. Here, the status of the patient may also be controlled (active versus inactive) and the patient may be grouped according to the provider or other indicia (e.g., particular medical study group, etc.). This tab also provides access to report cards and chart notes for the particular patient. Lastly, the Other tab includes basic functionality for setting up new groups although it will be appreciated that additional functionality may be added to this window.

In recapitulation, the present invention is a method and system to enable healthcare providers to utilize computers in chronic disease treatment, and more particularly to the management of chronic diseases in a manner that follows recognized standard-of-care recommendations (SOC) and links the patient to benefit opportunities such as medication samples and other benefits offered by pharmaceutical manufacturers and insurers.

It is, therefore, apparent that there has been provided, in accordance with the present invention, a method and apparatus for assisting in the management of chronic diseases. While this invention has been described in conjunction with preferred embodiments thereof, it is evident that many alternatives, modifications, and variations will be apparent to those skilled in the art. Accordingly, it is intended to embrace all such alternatives, modifications and variations that fall within the spirit and broad scope of the appended claims. The claims, as originally presented and as they may be amended, encompass variations, alternatives, modifications, improvements, equivalents, and substantial equivalents of the embodiments and teachings disclosed herein, including those that are presently unforeseen or unappreciated, and that, for example, may arise from applicants/patentees and others.

1. A method to enable healthcare professionals to utilize a computer system to provide chronic disease management capabilities to treat patients with a chronic disease according to a standard of care recommendation, comprising:
enabling, in accordance with pre-programmed code, collection and entry of patient parameters associated with the standard of care recommendation into a computer system during a visit;
storing the patient parameters in a database;
using entered patient parameters, automatically generating a patient report card, said report card indicating at least information pertaining to the patient parameters;
automatically printing, for the patient, a disease-management calendar showing a future visit and any lab date; and
automatically printing, for the patient, a complete medication schedule, wherein the schedule includes any changes to medication in accordance with the healthcare professional's recommendation.

2. The method of claim 1, further comprising the steps of using the computer system to identify patient parameters that depart from patient treatment goals, and indicating the same in a visual display.

3. The method of claim 2, further comprising the step of automatically printing a report card for the patient indicating progress toward treatment goals and a future checkup date.

4. The method of claim 1, further comprising the step of automatically linking the patient to appropriate samples at an office visit.

5. The method of claim 1, wherein the chronic disease includes diabetes and where the standard of care protocol is in accordance with patient-care requirements of the American Diabetes Association.

6. The method of claim 1, wherein the step of enabling collection and entry of patient parameters is accomplished via a plurality of pre-formatted interactive screens displayed on a computer interface.

7. The method of claim 1, wherein the step of enabling collection and entry of patient parameters is accomplished, at least in part, by access to an electronic medical record for the patient and extraction of data therefrom.

8. The method of claim 7, wherein the interactive screens further provide a steered entry process including fixed sequences and flashing entry prompts to direct a user to enter patient parameters.

9. The method of claim 8, wherein the steered entry process is facilitated by user selection from a plurality of pre-programmed options for at least one patient parameter.

10. The method of claim 7, wherein the interactive screens direct a user to input patient parameters relating to patient history, patient lifestyle, patient medications, patient self-monitored testing, and results of a physical examination of the patient.

11. The method of claim 7, wherein the interactive screens direct a user to input patient demographics, history, medications, physical findings, laboratory results, and other medical consultant visits.

12. The method of claim 1, wherein the step of automatically printing a disease-management calendar further includes dates for recommended tests and referrals.

13. The method of claim 1, further comprising the step of printing a provider chart note summarizing at least a portion of the patient parameters for inclusion in a written patient record.

14. The method of claim 1, further comprising the step reporting entered and missing required patient parameters.

15. The method of claim 1, further comprising the step of producing a diagnosis based on entered patient parameters and standard of care recommendations.

16. The method of claim 1, further comprising the step of providing suggested treatment considerations based on entered patient parameters and standard of care recommendations.

17. The method of claim 16, further comprising the step reporting departures from standard of care protocol treatment goals.

18. The method of claim 1, further comprising the step of providing warnings of drug contraindications and interactions based on entered patient parameters.

19. The method of claim 1, further comprising the step of providing recommendations to specialist consults based on entered patient parameters and standard of care recommendations.

20. The method of claim 1, wherein the patient report card is printed and given to patient at end of each visit, so that it may be reviewed with the patient at the time of the visit and the patient may bring it to the next visit, and where the report card includes a disease-management calendar showing next visits, next labs date, and any other recommended medical consults, a complete medication schedule including any changes recommended during the current visit, an indication of the level of control of the disease and associated treatment goals, and a patient checkout menu that includes a list of samples to be provided to the patient.

21. The method of claim 1, further comprising the step of providing system administration functionality and enabling a user of the system to customize the system to their preferences.

22. The method of claim 21, wherein the user is capable of customizing the system to administer at least the users, patient link information, treatment goal levels, lab testing frequencies, and patient visit frequencies.

23. The method of claim 21, further comprising the step of setting, in accordance with a user's input, at least one default value.

24. The method of claim 21, further comprising the step of administering the users of the system so as to control access thereto.

25. The method of claim 20, wherein the list of samples is determined as a function of a pre-programmed patient link table linking a medication to a textual output on the patient report card.

26. The method of claim 25, wherein the data stored in the patient link table can be altered by an administrator of the system.

27. The method of claim 1, wherein the default patient treatment goals can be altered by an administrator of the system.

28. The method of claim 1, wherein the default frequency of medical testing and medical professional visits can be altered by an administrator of the system.

29. The method of claim 1, further including the step of displaying, in accordance with at least one grouping parameter, a listing of patients for whom patient parameter data has been collected and stored in the system.

30. A method to enable healthcare professionals to provide patients with access to patient benefits during treatment of a chronic disease according to a standard of care protocol, comprising:
enabling, in accordance with pre-programmed code, collection and entry of patient parameters associated with the standard of care protocol into a computer system during a visit;
storing the patient parameters in a database;
using entered patient parameters, automatically identifying pharmaceutical benefits available to the patient and appropriate for the patient's treatment goals; and
using entered patient parameters, automatically generating a patient report card, said report card indicating at least information pertaining to the patient parameters.

31. The method of claim 30, further comprising the step of printing a checkout menu to identify needed samples to be made available to the patient.

32. The method of claim 30, further comprising the step of facilitating patient awareness and delivery of patient cost-saving benefits for medications and supplies the patient is currently taking.

33. The method of claim 30, further comprising the step of enabling patients to receive samples from a manufacturer by request.

34. The method of claim 33, wherein the step of enabling patients to receive samples includes printing, on the patient report card, a redeemable coupon for patients to return to the manufacturer in order to receive the sample.

35. An electronic medical records system for healthcare professionals, said system facilitating the delivery of uniform disease management services to patients with a chronic disease according to a standard of care recommendation, said system comprising:
an interface, operating in accordance with pre-programmed code, for collecting patient data associated with the standard of care protocol recommendation into the system during a visit;
a storage device for storing the patient data in at least one database;
a processor for compiling entered patient data and automatically printing a patient report card, said report card indicating at least information pertaining to the patient data;
automatically printing, for the patient, a disease-management calendar showing a future visit and any lab date; and
automatically printing, for the patient, a complete medication schedule, wherein the schedule includes any changes to medication in accordance with the healthcare professional's recommendation.

36. The system of claim 35, further comprising a checkout menu to identify needed samples to be made available to the patient.

37. The system of claim 35, further comprising an administration interface wherein at least one default parameter for a patient goal can be edited.
